SEQUENCE LISTING (22 sequences)

SEQ ID NO: 1 - protein, 121 aa, Homo sapiens
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKR

SEQ ID NO: 2 - protein, 121 aa, Macquaria novemaculeata
MSMLFYTLITAFLIGIQAEPHSESNVPAGHTIPQAHWTKLQHSLDTALRRVRSAPAVAIAARVAGQTRNITVDPRLFKKRRLRSPRVLFSTQPPPEAADTQDLDFEVGGAAPFNRTHRSKR

SEQ ID NO: 3 - protein, 120 aa, Sus scrofa
MSMLFYTITALLIGIQAEPHTESNVPAGHAIPQAHWTKLQHSLDTALRRAHSAPAGANSARVAGQTRNITVDPKLFKKRRLRSPRVLFSTQPPPVAADTQDPDLEASGAASFNRTHRSKR

SEQ ID NO: 4 - protein, 121 aa, Canis familiaris
MSMLFYTLITALLIGIRAEPHPESHVPAGHAIPHAHWTKLQHSLDTALRRARSAPAGAIAARVTGQTRNITVDPKLFKKRRLRSPRVLFSTHPPPVAADAQDLDLEAGSTASVNRTHRSKR

SEQ ID NO: 5 - protein, 121 aa, Rattus norvegicus
MSMLFYTLITAFLIGVQAEPYTDSNVPEGDSVPEAHWTKLQHSLDTALRRARSAPAEPIAARVTGQTRNITVDPKLFKKRRLRSPRVLFSTQPPPTSSDTLDLDFQAHGTISFNRTHRSKR

SEQ ID NO: 6 - protein, 121 aa, Mus musculus
MSMLFYTLITAFLIGVQAEPYTDSNVPEGDSVPEAHWTKLQHSLDTALRRARSAPTAPIAARVTGQTRNITVDPRLFKKRRLHSPRVLFSTQPPPTSSDTLDLDFQAHGTIPFNRTHRSKR

SEQ ID NO: 7 - protein, 128 aa, Homo sapiens
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRR

SEQ ID NO: 8 - protein, 138 aa, Homo sapiens
MSILFYVIFLAYLRGIQGNNMDQRSLPEDSLNSLIIKLIQADILKNKLSKQMVDVKENYQSTLPKAEAPREPERGGPAKSAFQPVIAMDTELLRQQRRYNSPRVLLSDSTPLEPPPLYLMEDYVGSPVVANRTSRRKR

SEQ ID NO: 9 - protein, 80 aa, Homo sapiens
MLPLPSCSLPILLLFLLPSVPIESQPPPSTLPPFLAPEWDLLSPRVVLSRGAPAGPPLLFLLEAGAFRESAGAPANRSRR

SEQ ID NO: 10 - protein, 5 aa, Artificial Sequence
  Synthetic sequence
XXRVX

SEQ ID NO: 11 - protein, 6 aa, Artificial Sequence
  Synthetic Sequence
XXRVXX

SEQ ID NO: 12 - protein, 7 aa, Artificial Sequence
  Synthetic Sequence
XXRVXXX

SEQ ID NO: 13 - protein, 8 aa, Artificial Sequence
  Synthetic Sequence
XXRVXXSX

SEQ ID NO: 14 - protein, 1159 aa, Homo sapiens
MAHRGPSRASKGPGPTARAPSPGAPPPPRSPRSRPLLLLLLLLGACGAAGRSPEPGRLGPHAQLTRVPRSPPAGRAEPGGGEDRQARGTEPGAPGPSPGPAPGPGEDGAPAAGYRRWERAAPLAGVASRAQVSLISTSFVLKGDATHNQAMVHWTGENSSVILILTKYYHADMGKVLESSLWRSSDFGTSYTKLTLQPGVTTVIDNFYICPTNKRKVILVSSSLSDRDQSLFLSADEGATFQKQPIPFFVETLIFHPKEEDKVLAYTKESKLYVSSDLGKKWTLLQERVTKDHVFWSVSGVDADPDLVHVEAQDLGGDFRYVTCAIHNCSEKMLTAPFAGPIDHGSLTVQDDYIFFKATSANQTKYYVSYRRNEFVLMKLPKYALPKDLQIISTDESQVFVAVQEWYQMDTYNLYQSDPRGVRYALVLQDVRSSRQAEESVLIDILEVRGVKGVFLANQKIDGKVMTLITYNKGRDWDYLRPPSMDMNGKPTNCKPPDCHLHLHLRWADNPYVSGTVHTKDTAPGLIMGAGNLGSQLVEYKEEMYITSDCGHTWRQVFEEEHHILYLDHGGVIVAIKDTSIPLKILKFSVDEGLTWSTHNFTSTSVFVDGLLSEPGDETLVMTVFGHISFRSDWELVKVDFRPSFSRQCGEEDYSSWELSNLQGDRCIMGQQRSFRKRKSTSWCIKGRSFTSALTSRVCECRDSDFLCDYGFERSSSSESSTNKCSANFWFNPLSPPDDCALGQTYTSSLGYRKVVSNVCEGGVDMQQSQVQLQCPLTPPRGLQVSIQGEAVAVRPGEDVLFVVRQEQGDVLTTKYQVDLGDGFKAMYVNLTLTGEPIRHRYESPGIYRVSVRAENTAGHDEAVLFVQVNSPLQALYLEVVPVIGLNQEVNLTAVLLPLNPNLTVFYWWIGHSLQPLLSLDNSVTTRFSDTGDVRVTVQAACGNSVLQDSRVLRVLDQFQVMPLQFSKELDAYNPNTPEWREDVGLVVTRLLSKETSVPQELLVTVVKPGLPTLADLYVLLPPPRPTRKRSLSSDKRLAAIQQVLNAQKISFLLRGGVRVLVALRDTGTGAEQLGGGGGYWAVVVLFVIGLFAAGAFILYKFKRKRPGRTVYAQMHNEKEQEMTSPVSHSEDVQGAVQGNHSGVVLSINSREMHSYLVS

SEQ ID NO: 15 - DNA, 22 bp, Artificial Sequence
  Synthetic Oligonucleotide
tgaagcccactggactaaactt

SEQ ID NO: 16 - DNA, 20 bp, Artificial Sequence
  Synthetic Oligonucleotide
aatctggaacatcgtatggg

SEQ ID NO: 17 - DNA, 25 bp, Artificial Sequence
  Synthetic Oligonucleotide
gagatccactagttctagcctcgag

SEQ ID NO: 18 - DNA, 23 bp, Artificial Sequence
  Synthetic Oligonucleotide
cccacacactgacactgtcacac

SEQ ID NO: 19 - DNA, 25 bp, Artificial Sequence
  Synthetic Oligonucleotide
ttataacaccctgttacgtatagcc

SEQ ID NO: 20 - DNA, 25 bp, Artificial Sequence
  Synthetic Oligonucleotide
tatctctgaccagagtcatccttag

SEQ ID NO: 21 - DNA, 25 bp, Artificial Sequence
  Synthetic Oligonucleotide
aaagagaagctgtgctatgttgctc

SEQ ID NO: 22 - DNA, 25 bp, Artificial Sequence
  Synthetic Oligonucleotide
gcatagaggtctttacggatgtcaa

(19) United States
(12) Patent Application Publication
(10) Pub. No.: US 2013/0115222 A1
(43) Pub. Date: May 9, 2013

(54) METHODS OF LIMITING MICROVASCULAR DAMAGE FOLLOWING ACUTE MYOCARDIAL ISCHEMIA

(71) Applicants: Barbara Hempstead, New York, NY (US); Chia-Jen Siao, New York, NY (US)

(72) Inventors: Barbara Hempstead, New York, NY (US); Chia-Jen Siao, New York, NY (US)

(73) Assignee: Cornell University, Ithaca, NY (US)

(21) Appl. No.: 13/704,404

(22) PCT Filed: Jun. 15, 2011
(86) PCT No.: PCT/US11/40447

§ 371 Date: Jan. 25, 2013

Related U.S. Application Data
(60) Provisional application No. 61/397,663, filed on Jun. 15, 2010.

(51) Int. Cl. A61K 39/395; A61K 31/713

(52) U.S. Cl. CPC A61K 39/3955; A61K 31/713
USPC 424/158.1; 514/44 A; 514/8.4; 424/172.1; 514/1.1

(57) ABSTRACT

This disclosure has identified a new ligand-receptor system, proNGF and p75NTR/SorCS2, which is found to be involved in the microvascular functions of the heart. This disclosure provides methods for limiting microvascular damage following acute myocardial ischemia based on administration of an antagonist of this newly identified system, thereby promoting myocardial recovery.

### CROSS REFERENCE TO RELATED APPLICATION

This application claims the benefit of priority of U.S. Provisional Application No. 61/397,663, filed Jun. 15, 2010, the entire content of which is incorporated herein by reference.

### STATEMENT REGARDING FEDERALLY SPONSORED RESEARCH OR DEVELOPMENT

This invention was made with Government Support under Contract Nos. PO1HL04603 and R01 NS030687, awarded by the National Institutes of Health. The Government has certain rights in this invention.

### FIELD OF THE DISCLOSURE

This invention relates to the identification of a ligand-receptor system involved in the microvascular functions of the heart, and to the manipulation of the system to prevent or limit microvascular dysfunction and damage following acute myocardial ischemia, thereby promoting myocardial recovery.

### BACKGROUND ART

Treatments of acute myocardial infarction focus primarily on limiting the duration of ischemia, using angioplasty or thrombolysis to establish reperfusion. However, even with reestablishment of macrovascular patency, a significant proportion of patients exhibit microvascular damage in the infarcted tissue leading to tissue fibrosis, impaired cardiac contractility, and ultimately to a higher incidence of death (Eltzschig et al., Brit. Med. Bulletin, 70:71-86 (2004)). To date, the specific pro-inflammatory cytokines that mediate microvascular dysfunction or apoptosis following cardiac ischemia are unknown, and identification of such locally produced factor(s) would provide important potential therapeutic targets to limit microvascular dysfunction, and promote myocardial recovery following reperfusion.

Nerve growth factor (NGF) mRNA is rapidly induced following cardiac ischemia-reperfusion in rodents (Hiltunen et al., Journal of Pathology, 194(2):247-253 (2001); Hasan et al., Brain Research, 1124(1):142-154 (2006)). NGF, the prototypic member of the neurotrophin family which also includes brain-derived neurotrophic factor (BDNF), and neurotrophin-3 (NT-3), binds to the receptor tyrosine kinase TrkA to mediate cell survival and differentiation (Reichardt, Philos Trans R Soc Lond B Biol Science, 361(1473):1545-1564 (2006)). Neurotrophins are initially synthesized as precursors, or proneurotrophins, which can be proteolytically cleaved to release the C-terminal domain, or mature neurotrophin. Recent studies show that proNGF is not an inactive precursor, but acts as a signaling ligand, distinct from its mature counterpart, to mediate neuronal cell death (Lee, Science, 294: 1945-48 (2001), and reviewed in Hempstead, Neurotoxicity Research, 16: 255-60 (2009)). The pro-apoptotic action of proNGF requires the expression of a distinct receptor p75NTR, which encodes an intracellular death domain, and a co-receptor, sortilin (Nykjaer et al., Nature, 427(6977):843-848 (2004); Jansen et al., Nat Neuroscience, 10(11):1449-1457 (2007)).

In the cardiovascular system, the functions of the related neurotrophins BDNF and NT-3 have been established in promoting vascular integrity and regulating cardiac septation (Donovan et al., Nat Genet, 14(2):210-213 (1996); Donovan et al., American Journal of Pathology, 147(2): 09-324 (1995); and reviewed in Caporali et al., Physiol Review, 89(1):279-308 (2009)). However, the actions of cardiac NGF are less well characterized. NGF is synthesized by cardiomyocytes and cardiac vascular smooth muscle cells, and promotes sympathetic innervation (Glenbova and Ginty, J. Neurosci 24: 743 (2004)). In addition, myocyte-derived NGF can acutely modulate synaptic transmission between sympathetic neurons and cardiac myocytes (Lockhart et al., Journal of Neuroscience, 17(24):9573-9582 (1997)), in part by altering the firing properties of these neurons (Luther et al., Journal of Neurophysiology, 96(2):946-958 (2006)). Indeed, NGF-deficient (NGF^{-/-}) mice die perinatally of presumed innervation defects without overt abnormalities of the cardiovascular system (reviewed in Caporali et al., Physiol Review, 89(1):279-308 (2009)).

ProNGF and p75NTR are expressed at low to undetectable levels in most adult tissues, but are rapidly induced following acute injury and promote apoptosis, best studied in the central and peripheral nervous systems (reviewed in Hempstead, Neurotox Research, 16(3):255-260 (2009)). Although no studies have evaluated proNGF levels following cardiac ischemia, the level of NGF mRNA increases within hours following cardiac ischemia/reperfusion, and elevated protein levels are maintained for several days before returning to baseline (Hiltunen et al., Journal of Pathology, 194(2):247-253 (2001)). In addition, local expression of p75NTR is increased in endothelial cells and vascular smooth muscle cells following acute aortic injury (Donovan et al., American Journal of Pathology, 147(2): 09-324 (1995)), and p75NTR activation promotes vascular smooth muscle cell apoptosis (Wang et al., Am J Pathology 157: 1247-58, 2000) and endothelial cell apoptosis (Kim et al., Journal of Biological Chemistry, 279(32):33538-33546 (2004)). p75NTR deficiency (p75NTR^{-/-}) results in reduced apoptosis of vascular smooth muscle cells following carotid artery injury, suggesting a role for locally produced neurotrophins in regulating this vascular response (Kraemer, Circ Res, 91(6):494-500 (2002)). No studies to date have examined sortilin, or other members of the sortilin family members including SorCS1, SorCS2, SorCS3, in the vasculature.

Endothelial cells depend on complex and reciprocal interactions with pericytes during vascular development, and pericytes maintain microvascular structure and function in the adult vasculature (reviewed in Gaengel et al., Arterioscler Thromb Vasc Biol, 29:630-638 (2008)). Disruption of endothelial:pericyte communication leads to vascular hemorrhage and embryonic lethality, best exemplified by mice deficient in Pdgfb or Pdgfrb, which fail to recruit pericytes to specific vascular beds (Lindahl et al., Science, 277(5323):242-245 (1997); Hellstrom et al., Development 126:3047-55 (1999)). In adult mice, TGFβ and bone morphogenetic proteins play critical roles in maintaining pericyte survival and promoting microvascular integrity, as genetic deletion of Bmp1a in smooth muscle cells leads to cardiac dysfunction under hypoxic conditions, and blockade of TGFβ leads to abnormal vascular permeability (El-Bizri et al., Circ Res 102(3):380-388 (2008); Walshe et al., PLoS One 4(4):1-16 (2009)). In summary, dysregulation of endothelial cell:pericyte crosstalk during maturation of the cardiac microvasculature leads to cardiac dysfunction later on in life.

### SUMMARY OF THE DISCLOSURE

The inventors have identified a new ligand-receptor system, proNGF and p75NTR/SorCS2, to be involved in the microvascular functions of the heart. Accordingly, this disclosure provides methods for limiting microvascular damage following acute myocardial ischemia based on administration of an antagonist of this newly identified system, thereby promoting myocardial recovery.

One aspect of this disclosure is directed to a method of limiting microvascular damage following acute myocardial ischemia in a subject by administering a proNGF antagonist. A proNGF antagonist can include a neutralizing antibody that binds specifically to proNGF, an aptamer, an oligopeptide, a small molecule compound, or a nucleic acid molecule which reduces the level or activity of a proNGF mRNA, for example.

Another aspect of this disclosure is directed to a method of reducing microvascular dysfunction and the associated damage following acute myocardial ischemia by administering a SorCS2 antagonist. A SorCS2 antagonist can include an antibody, an aptamer, an oligopeptide, a small molecule compound, or a nucleic acid molecule which reduces the level or activity of the SorCS2 mRNA.

An antagonist or a cocktail of antagonists can be administered to a subject as soon as practical after acute myocardial ischemia (AMI) occurs. In some embodiments, an antagonist or a cocktail of antagonists is administered to the subject within 48 hours. In a specific embodiment, administration is achieved within 2-6 hours of the AMI.

An antagonist or a cocktail of antagonists can be combined with a pharmaceutically acceptable carrier for administration, and can be given to a subject via standard routes including ingestion, injections via an intravenous, intraperitoneal, subcutaneous, transdermal, intramuscular, intranasal, or sublingual route, or via catheter delivery at the time of percutaneous intervention or during an open heart surgery.

### BRIEF DESCRIPTION OF THE DRAWINGS

FIG. 1. Schematic of Ngf gene targeting with non-furin-mutated Ngf-HA allele. Inset, Southern blot.

FIG. 2. Expression of proNGF and its receptor p75NTR were upregulated following ischemia-reperfusion injury. (a) H&E stained sections from LV of uninfarcted (left panels) and infarct-reperfused wtNGF-HA/+ mice at a region of peri-infarct (right panels). Note the increased numbers of infiltrating cells in the injured myocardium compared with control section. (b) Anti-HA immunohistochemistry shows the increased NGF-HA reactivity in both cardiomyocyte and in infiltrating cells (inset). (c) Use of a proNGF-specific antibody confirms the HA antibody results, and further shows that the increased NGF species in the injured myocardium and in infiltrating cells (arrows) was proNGF, not mature NGF. (d) Sortilin was expressed on arteriolar smooth muscle cells, and was not upregulated post-I/R. (e) p75NTR and TH double immunofluorescence shows dystrophic sympathetic nerves in the I/R-injured hearts, compared with normal processes in the control uninjured section. (f) p75NTR was expressed on PDGFRβ+ pericytes in the peri-infarct regions of I/R-injured hearts, but not on pericytes of uninjured myocardium. (g) SorCS2 was expressed on arteriolar smooth muscle cells. (h) SorCS2 was expressed by a subset of PDGFRβ+ pericytes in the peri-infarct region. Scale bars: (a) and (b), 100 μm; (c), 40 μm; (d)-(g), 20 μm.

FIG. 3. (a) IP-WB from 293T cells showing interactions between proNGF and sortilin and SorCS2. Note that proNGF co-immunoprecipitated with both sortilin and SorCS2. (b) Immunoprecipitation-Western blot analysis of proNGF or NGF expression. Left, lysates of P0 NGF+/+ and proNGF-HA/+ hearts (3 hearts pooled per lane) demonstrates proNGF-HA (˜32 kD) but not mature NGF (˜13 kD) in the proNGF-HA/+ but not the NGF+/+ hearts. Right, adult brain lysates demonstrate that mature NGF-HA, but not proNGF-HA is detected in wtNGF-HA/+ brain extracts, whereas proNGF-HA is the most prominent isoform in proNGF-HA/+ brain extracts.

FIG. 4. Schematic of Prongf-HA gene targeting. DT, diphtheria toxin cassette. The dark bars indicate Southern blot probe sequence and expected product sizes. Inset, Southern blot analysis for embryonic cells positive for the replaced Prongf-HA allele.

FIG. 5. Older proNGF-HA/+ hearts exhibited myocardial thinning and dilation, fibrosis, and histiocyte infiltration. (a-f) H&E stained sections of 8 mo NGF^{+/+} (a, d), NGF^{+/-} (b, e), and proNGF-HA/+ (c, f) hearts. (g-l) Masson's trichrome analysis of 8 mo NGF^{+/+} (g), NGF^{+/-} (h), and proNGF-HA/+ (i) hearts. Red, myocardium. Black, nuclei. Blue, collagen matrix. Note that the blue collagen deposition is normally observed around large arterioles. However, in the proNGF-HA/+ hearts, the blue collagen is extensively laid down in place of myocyte tissue. CD68 immunofluorescent analysis of 6 mo NGF (j), NGF^{+/-} (k), and proNGF-HA/+ (l) hearts, showing increased infiltration by histiocytes including monocytes and macrophages. Scale bars, (a-c), 2 mm; (d-l), 100 μm.

FIG. 6. TEM images of 4 mo NGF^{+/+}, NGF^{+/-}, and proNGF-HA/+ hearts. At this age the proNGF-HA/+ myocardium showed many regions of necrosis, presumably due to microinfarcts in the vasculature. However, NGF^{+/+} and NGF^{+/-} myocardial tissues still appeared normal. Arrowheads, normal endothelial cells. Arrow, disrupted endothelial cell with fragmented cell membrane. Scale bar, 2 μm.

FIG. 7. Echocardiographic analysis of NGF^{+/+}, proNGF-HA/+, and p75^{-/-}; proNGF-HA/+ mice. (a) Examples of M-mode traces from 2 mo (left) and 4 mo (right) NGF^{+/+} (top), proNGF-HA/+ (middle), and p75^{-/-}; proNGF-HA/+ (bottom) hearts. (b) Progressive cardiac dysfunction in proNGF-HA/+ mice (solid red squares) compared with normal NGF^{+/+} (blue diamonds) mice. ***, P<0.005, t-test. Genetic deletion of p75, the receptor for proNGF, in the p75^{-/-}; proNGF-HA/+ mice, rescued the cardiac deficit (open red squares). NS, not significant compared with NGF^{+/+} mice. Sortilin deletion does not rescue the dilated cardiomyopathy phenotype (filled green triangles). #, P<0.05 sort^{-/-}; proNGF-HA/+ compared with NGF+/+, t-test. % FS, fractional shortening (see text). Solid blue and red, and dashed red and green lines, linear regression curve for each genotype over time. N_{(NGF+/+)}=18. N_{(proNGF-HA/+)}=31. N_{(p75-/-; proNGF-HA/+)}=10. N_{(sort-/-; proNGF-HA/+)}=10. (c) Kaplan-Meier survival graph of NGF^{+/+} and proNGF-HA/+ mice over age of mice.

FIG. 8. Young proNGF-HA/+ cardiac microvascular endothelial cells exhibited an activated phenotype. (a-c) H&E histology of 1 mo NGF^{+/+} (a), NGF^{+/-} (b), and proNGF-HA/+ (c) hearts. (d-f) Transmission electron micrographs of 1 mo NGF^{+/+} (d), NGF^{+/-} (e), and proNGF-HA/+ (f) hearts show activated endothelial cells in proNGF-HA/+ cardiac microvasculature. Arrowheads, normal endothelial cells with smooth cell membrane ensheathing plasma or a red blood cell. Arrow, activated endothelial cell with thickened and filamentous cell membranes containing numerous vacuoles. In the proNGF-HA/+ myocardium, extensive (˜50% of vessels examined) activation of microvascular endothelial cells were found as well as edematous regions where vessels did not contact neighboring myocardium (asterisk; as opposed to sectioning artifact visible in panel (e)). Note that, in all genotypes, the myocardium was normal and healthy in appearance at this time point. Scale bars, (a-c), 100 μm; (d-f), 2 μm.

FIG. 9. Microvascular endothelial cell activation and aberrant platelet deposition during maturation of the cardiac vasculature in NGF+/pro mice. (a-c) CD41, a platelet/megakaryocyte marker, shows numerous platelet aggregates in proNGF-HA/+ (b) hearts compared with NGF^{+/+} (a) in P9 myocardium. p75^{-/-}; proNGF-HA/+ vessels did not trap platelets (c). (d-f) ICAM-1 upregulation indicates endothelial activation in the proNGF-HA/+ (e) vessels, not in the NGF^{+/+} (d) or p75^{-/-}; proNGF-HA/+ (f) vessels, at P9. (g-i) FITC-dextran (70 kD) accumulation, indicating loss of ability of vessels to clear the macromolecule, and extravasation, indicating microvascular edema and loss of integrity, in proNGF-HA/+ myocardium (h) was rescued by p75-loss (i). (j-k) Quantification of CD41 and ICAM-1 immunofluorescence. Scale bar, 100 μm.

FIG. 10. Expression of proNGF receptors during development contributed to eventual cardiac microvascular dysfunction. E17.5 or P0 embryos were analyzed for p75NTR and SorCS2 immunoreactivity. p75NTR was not expressed by isolectin B4+ endothelial cells (a), but was expressed by a subset of PDGFRβ+ pericytes (b). (c) SorCS2 was expressed by a small subset of PDGFRβ-immunopositive pericytes. (d) p75NTR and SorCS2 were co-expressed only by a small number of pericytes at E20. Scale bars, 10 μm.

FIG. 11. Autopsy sections from human hearts in the left ventricular wall, or in the peri-infarct area of individuals dying within 3 days of a myocardial infarction. Immunoreactivity with the indicated antibody was detected by a red-brown reaction product. This figure shows proNGF was upregulated massively in autopsy specimens from those dying of a heart attack, but not in those that die of other causes. In addition, p75 was upregulated in pericytes and the vascular smooth muscle cells of small arterioles.

FIG. 12. Prodomain of proNGF from relevant animal models: human (SEQ ID NO: 1), macque monkey (SEQ ID NO: 2), pig (SEQ ID NO: 3), dog (SEQ ID NO: 4), rat (SEQ ID NO: 5), and mouse (SEQ ID NO: 6).

FIG. 13. Human proneurotrophin prodomains. The prodomains of human proNGF, proBDNF, proNT-3 and proNT4 are set forth in SEQ ID NOS: 1, 7, 8 and 9, respectively.

### DETAILED DESCRIPTION

The inventors have identified a new ligand-receptor system involved in the microvascular functions of the heart, which can be exploited to prevent microvascular dysfunction in the ischemic heart or limit microvascular damage following acute myocardial ischemia, thereby promoting myocardial recovery.

More specifically, the inventors have determined that both the proNGF ligand and its receptors p75NTR and SorCS2, a sortilin family member, are induced in the infarcted murine heart. The inventors have generated a knock-in mouse in which one allele expresses a cleavage-resistant form of proNGF under the control of the endogenous Ngf promoter, and the other non-targeted allele expresses NGF (termed proNGF-HA/+ mice). These mice are viable, but develop a dilated cardiomyopathy in early adulthood, leading to mortality by 6 to 8 months of age. The inventors have shown that pericytes ensheathing cardiac capillaries express p75NTR and SorCS2, but not sortilin, during late embryogenesis. The inventors have also shown that proNGF-expressing animals deficient in p75NTR do not develop cardiomyopathy, whereas proNGF-expressing animals deficient in sortilin are not rescued from this phenotype. In addition, the inventors have demonstrated that in the proNGF-HA/+ mice, endothelial cells are activated, leading to increased vascular permeability.

Without intending to be bound by any particular theory, it is believed that induction of proNGF and p75NTR in the heart of the proNGF-HA/+ mice act upon pericytes to promote dysfunction of microvascular endothelial cells, leading to an activated endothelium and subsequent cardiomyopathy; and SorCS2, which binds to proNGF, functions as a co-receptor with p75NTR to alter pericyte function.

In accordance with the present disclosure, this ligand-receptor system (proNGF-p75NTR/SorCS2), newly identified by the inventors, can be manipulated to reduce, limit or prevent microvascular dysfunction and microvascular damage in the heart following acute myocardial ischemia.

### Microvascular Dysfunction and Damage

Acute myocardial ischemia (or AMI) is commonly known as heart attack, which occurs when blood supply to a part of the heart is interrupted. The resulting oxygen shortage can cause damage or death (or “infarction”) of heart muscle tissue if left untreated for a period of time. Prior to this disclosure, treatments of acute myocardial infarction focus primarily on reestablishment of blood flow in coronary arteries. However, even with reestablishment of macrovascular patency, a significant proportion of patients exhibit microvascular damage in the infarcted tissue that leads to chronically impaired heart function and disability, and a higher incidence of death (Eltzschig et al., Brit. Med. Bulletin, 70:71-86 (2004)). In accordance with this disclosure, however, myocardial recovery following reperfusion can be improved by preventing or otherwise limiting the microvascular dysfunction or damage through the use of an antagonist of the proNGF-p75NTR/SorCS2 system.

The term “microvascular” as used herein refers to small blood vessels, such as arterioles, capillaries and venules. Such small blood vessels are generally composed of two cell types: endothelial cells, which form the inner vessel wall, and pericytes, which form a layer around the endothelial tube (Gaengel et al., Arterioscler Thromb Vasc Biol 29: 630-638, 2009).

The term “microvascular dysfunction” refers to abnormalities in the structure and/or function of small blood vessels. Microvascular dysfunction can be assessed and determined by analyzing cardiac contractility, vascular permeability, electromicroscopy of microvasculature, and analysis of the levels of endothelial and pericyte markers (such as CD-31, isolectin B4, PDGFRβ and NG2), among others.

The term “microvascular damage” refers to the loss or compromise of the structural integrity and/or function of small blood vessels as a result of microvascular dysfunction.

### Antagonists of the proNGF-p75NTR/SorCS2 Ligand-Receptor System

As disclosed herein, administration of an antagonist of the proNGF-p75NTR/SorCS2 ligand-receptor system can reduce or limit, or event completely prevent, microvascular dysfunction and the associated damage following acute myocardial ischemia.

The term “antagonist” as used herein, refers to a molecule that inhibits the expression level of a component of the proNGF-p75NTR/SorCS2 ligand-receptor system on pericytes (“expression antagonist”); or alternatively, inhibits the interaction or binding between the components of the proNGF-p75NTR/SorCS2 ligand-receptor system expressed on pericytes (“binding antagonist”), thereby reducing the amount, formation, function, and/or downstream signaling of this ligand-receptor system.

A molecule is considered to inhibit the expression level of a component of the proNGF-p75NTR/SorCS2 system if the molecule causes a significant reduction in the expression (either at the level of transcription or translation) of the component. Similarly, a molecule is considered to inhibit the binding between the components of the proNGF-p75NTR/SorCS2 ligand-receptor system if the molecule causes a significant reduction in the binding between the components and the ligand-receptor complex formed, which causes a significant reduction in downstream signaling and functions mediated by the ligand-receptor system, e.g., activated endothelial cells and increased vascular permeability. A reduction is considered significant, for example, if the reduction is at least about 25%, and in some embodiments at least about 50%, and in other embodiments at least about 90%.

A binding antagonist can act in two ways. A binding antagonist can compete with the ligand proNGF for the receptors thereby interfering with, blocking or otherwise preventing the binding of proNGF to p75NTR and/or SorCS2. This type of antagonist, which binds the receptor but does not trigger the expected signal transduction, is also known as a “competitive antagonist” and can include, for example, an oligopeptide designed based on a proNGF sequence, or an antibody directed to SorCS2. Alternatively, a binding antagonist can bind to and sequester the ligand, proNGF, with sufficient affinity and specificity to substantially interfere with, block or otherwise prevent binding of proNGF to p75NTR and/or SorCS2. This type of antagonist is also known as a “neutralizing antagonist”, and can include, for example, an antibody or aptamer directed to proNGF which binds specifically to proNGF.

An antagonist can also be characterized based on the target molecule which the antagonist is intended to antagonize. For example, a proNGF antagonist refers to a molecule which inhibits or reduces the expression of proNGF; or interferes with, blocks or otherwise prevents the interaction or binding of proNGF to p75NTR and/or SorCS2. On the other hand, a SorCS2 antagonist refers to a molecule which inhibits or reduces the expression of SorCS2; or interferes with, blocks or otherwise prevents the interaction between SorCS2 and proNGF and/or p75NTR.

### ProNGF Antagonist

In one embodiment, this disclosure provides a method of reducing microvascular dysfunction and the associated damage following acute myocardial ischemia by administration of a proNGF antagonist.

As disclosed herein, a proNGF antagonist can be a neutralizing antibody that is specific for proNGF, an aptamer, an oligopeptide, a small molecule compound, or a nucleic acid molecule which reduces the level or activity of a proNGF mRNA, for example.

In a specific embodiment, a proNGF antagonist is a neutralizing antibody that is specific for proNGF.

In this disclosure, a molecule (such as an antibody or aptamer) that is specific for proNGF is a molecule that binds with substantially greater affinity, and in some embodiments, binds nearly exclusively to proNGF, relative to a mature NGF and other proneurotrophins. By “substantially greater affinity” it is meant that the binding affinity (Kd) of a molecule for a proNGF is at least 5 fold, 10 fold, 50 fold, 100 fold, or 1000 fold or greater, of the binding affinity of the molecule for the mature NGF or other proneurotrophins.

In one particular embodiment, an antibody specific for proNGF is an antibody directed to the prodomain of proNGF.

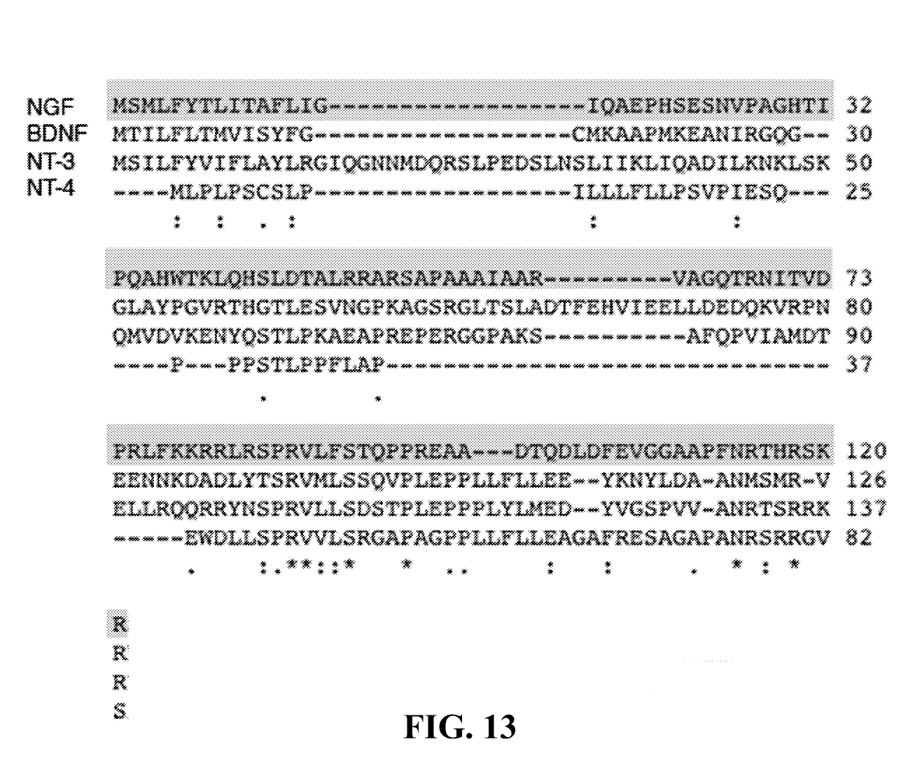

The prodomain of proNGF is clearly distinct from those of other proneurotrophins (FIG. 13), making it unlikely that antibodies raised against the prodomain of a proNGF will cross-react with other proneurotrophins, or with mature NGF. Where appropriate, the specificity of an anti-proNGF antibody can be confirmed by using assays known in the art, including those described in more detail in the examples hereinbelow.

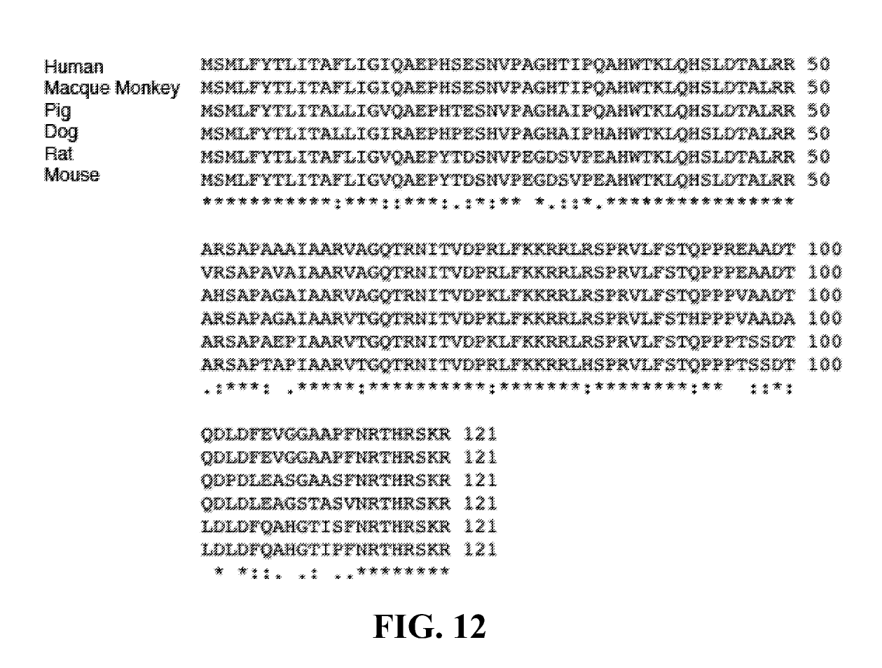

The pro-domain of proNGF is highly conserved across species. As shown in FIG. 12, significant regions of identity are present within the pro-domain of NGF from human, macque monkey, pig, dog, rat, and mouse, enabling the generation of a spectrum of antibodies to the prodomain directed to different regions, motifs, tertiary structures, or epitopes of the prodomain.

In certain embodiments, a proNGF-specific antibody is specifically directed to motifs or epitopes within the prodomain, including contiguous sequences of amino acids within the prodomain that are highly conserved across species as shown in FIG. 12.

The term “antibody” as used herein includes intact immunoglobulin molecules, as well as molecules that include an antibody hypervariable region that binds specifically to an intended antigen, with or without an antibody constant region. The hypervariable region can include an entire antibody variable region. Thus, an antibody molecule that includes an antibody hypervariable region can be an intact antibody molecule, antibody fragments (including single chain antibodies) which retain the antigen binding specificity of intact antibodies, as well as chimeric and humanized antibodies. The antibody can be polyclonal or monoclonal, and can be of any class of immunoglobins, such as: IgG, IgM, IgA, IgD or IgE, and the subclass thereof.

Suitable antibodies can be produced in a non-human mammal, including for example, rabbits, rats, mice, horses, goats, camels, or primates. Monoclonal antibodies produced from a non-human mammal can be humanized to reduce the immunogenicity for use in humans following techniques documented in the art. For example, to humanize a monoclonal antibody raised in mice, one approach is to make mouse-human chimeric antibodies having the original variable region of the murine mAb, joined to constant regions of a human immunoglobulin. Chimeric antibodies and methods for their production are well known in the art. See, e.g., Cabilly et al., European Patent Application 125023 (published Nov. 14, 1984); Taniguchi et al., European patent Application 171496 (published Feb. 19, 1985); Morrison et al., European Patent Application 173494 (published Mar. 5, 1986); Neuberger et al., PCT Application WO 86/01533, (published Mar. 13, 1986); all of which are incorporated herein by reference. Alternatively, humanized antibodies can be made to by including constant regions of a human immunoglobulin, and additionally, substituting framework residues of the variable regions of a non-human antibody with the corresponding human framework residues, either leaving the non-human CDRs substantially intact, or even replacing the CDR with sequences derived from a human genome. See, e.g., Maeda et al., Hum. Antibod. Hybridomas 2: 124-134, 1991, and Padlan, Mol. Immunol. 28: 489-498, 1991. As an additional alternative, human antibodies can be produced from transgenic animals (e.g., transgenic mice) whose immune systems have been altered to correspond to human immune systems. An example of such a mouse is the so-called XenoMouse™ (Abgenix, Freemont, Calif.), described by Green, “Antibody Engineering via Genetic Engineering of the Mouse: XenoMouse Stains are a Vehicle for the Facile Generation of Therapeutic Human Monoclonal Antibodies,” J. Immunol. Methods 10; 231(1-2):11-23(1999).

In another specific embodiment, a proNGF antagonist is an aptamer that binds specifically to proNGF.

Aptamers are molecules, either nucleic acid or peptide, that bind to a specific target molecule. Nucleic acid aptamers are generally short strands of DNA or RNA that have been engineered through repeated rounds of in vitro selection known as SELEX (systematic evolution of ligands by exponential enrichment) to bind to various molecular targets. Peptide aptamers can be selected using various systems, most frequently through the yeast two hybrid system. Peptide aptamers generally consist of a variable peptide loop (typically composed of ten to twenty amino acids), attached at both ends to a protein scaffold. This double structural constraint greatly increases the binding affinity of the peptide aptamer to levels comparable to an antibody.

In still another specific embodiment, a proNGF antagonist is an oligopeptide or a small molecule compound that binds to the receptors of proNGF (i.e., the p75 receptor and/or SorCS2) thereby blocking the binding of proNGF to its receptors, but does not lead to the downstream signaling or biological activity triggered by binding of proNGF to p75NTR/SorCS2.

Such small molecules and oligopeptides can be discovered by methods well known in the art. Typically, discovering such molecules involves providing a cell that expresses p75NTR and/or SorCS2, providing a small molecule or oligopeptide to be tested, and determining whether the small molecule or oligopeptide to be tested binds to p75NTR and/or SorCS2 and, optionally, results in the biological activity caused by binding of a proNGF to p75NTR/SorCS2. If the molecule binds with high affinity to p75NTR and/or SorCS2, it is a candidate for use in the present method to limit microvascular damage. If the molecule binds to p75NTR and/or SorCS2 with high affinity and blocks binding of proNGF to p75NTR/SorCS2, it is a stronger candidate. If, in addition to blocking binding, the molecule also fails to cause the biological activity expected from activating p75NTR/SorCS2, e.g. to activate endothelial cells, the molecule is a candidate for pre-clinical or clinical trials.

The oligopeptide has at least approximately four amino acid residues, and in some embodiments at least approximately five amino acid residues, and in other embodiments at least approximately six amino acid residues. The maximum number of amino acid residues is not important, as long as the oligopeptide has the desirable properties mentioned above. The oligopeptide may be linear or cyclic.

Some examples of oligopeptides include:

(SEQ ID NO: 10)
S/T-P/S-R-V-(Z)z
(SEQ ID NO: 11)
S/T-P/S-R-V-L/M/V-(Z)z
(SEQ ID NO: 12)
S/T-P/S-R-V-L/M/V-F/L-(Z)z
(SEQ ID NO: 13)
S/T-P/S-R-V-L/M/V-F/L-S-(Z)z

wherein Z represents any alpha amino acid and z represents any number from 0 to approximately 20, preferably from 0 to approximately 10, and more preferably from 0 to approximately 5. Any of these oligopeptides may be cyclic.

Small molecules include organic compounds, organometallic compounds, salts of organic and organometallic compounds, saccharides, amino acids, and nucleotides. Small molecules typically have molecular weights less than approximately 1000 Daltons, in some embodiments less than 800 Daltons. Small molecules include compounds that are found in nature as well as synthetic compounds.

In another specific embodiment, a proNGF antagonist administered is a nucleic acid molecule which reduces the level or activity of a proNGF mRNA. Such nucleic acid molecule includes an antisense RNA, a siRNA, a miRNA (or “microRNA”) or a transgene which codes for and is capable of expressing any such RNA molecule in the target tissue of a recipient. An antisense RNA is an RNA molecule that is complementary to endogenous mRNA and blocks translation from the endogenous mRNA by forming a duplex with the endogenous mRNA. siRNAs are small (typically 20-25 nucleotides in length) double-stranded RNAs which are known to be involved in the RNA interference pathway and interfere with the expression of a specific gene. Given the sequence of a target gene, siRNAs can be designed, and made either synthetically or in cells from an exogenously introduced vector (e.g., a plasmid) to achieve suppression of expression of a gene of interest. Similar to siRNAs, miRNAs are also small RNA molecules (generally about 21-22 nucleotides) that regulate gene expression. miRNAs are processed from long precursors transcribed from non-protein-encoding genes, and interrupt translation through imprecise base-pairing with target mRNAs. miRNA can be designed and introduced to cells or tissues to target and suppress the expression of a gene of interest (proNGF, SorCS2 or p75NTR) using techniques documented in the art. Modulation of miRNA can be accomplished by viral-mediated delivery of pro-miRNA or decoymiR or by delivery in plasma (as examples, Cordes K R, et al, Nature 460:705 (2009); Caporali A, et al., Circulation 123:282, (2011); Castoldi, M, J., Clin Invest. 121:1386 (2011); Vickers, K C et al., Nat Cell Biol 13: 423 (2011)).

### SorCS2 Antagonist

In a further embodiment, this disclosure provides a method of reducing microvascular dysfunction and the associated damage following acute myocardial ischemia by administration of a SorCS2 antagonist.

As disclosed herein, a SorCS2 antagonist can be an antibody, an aptamer, an oligopeptide, a small molecule compound, or a nucleic acid molecule which reduces the level or activity of the SorCS2 mRNA.

In a specific embodiment, a SorCS2 antagonist is an antibody that binds specifically to SorCS2 and inhibits the interaction of SorCS2 with proNGF and/or p75NTR. An antibody that is specific for SorCS2 is an antibody that binds with substantially greater affinity, and in some embodiments, binds nearly exclusively to SorCS2, relative to other members of the sortlin family such as sortlin. By “substantially greater affinity” it is meant that the binding affinity of an antibody for SorCS2 is at least 5 fold, 10 fold, 50 fold, 100 fold, or 1000 fold or greater, of the binding affinity of the antibody for other members of the sortlin family.

In one particular embodiment, a SorCS2-specific antibody is directed to the ectodomain of SorCS2 (amino acids 20-1078 of human SorCS2). In certain embodiments, a SorCS2-specific antibody is specifically directed to specific motifs or epitopes within the ectodomain, such as the cystein-rich domain (amino acid residues 611-750 of human SorCS2), or the 10 bladed propeller domains (amino acids 45-610). The amino acid sequence of human SorCS2 is set forth in SEQ ID NO: 14 (Accession No. NP_{—}065828).

Similar to proNGF antagonists as described above, SorCS2 antagonists are not limited to antibodies, but also include nucleic acid or peptide aptamers that bind specifically to SorCS2 and inhibit its interaction with proNGF and/or p75NTR, oligopeptides or small molecule compounds that block the interaction of SorCS2 with proNGF and/or p75NTR, as well as nucleic acid molecules (such as antisense, siRNA, or miRNAs) which reduce the level or activity of the SorCS2 mRNA.

### Cocktails of Antagonists

Also provided herein is a cocktail of more than one antagonist molecule, which is also suitable for administration. The cocktail may, for example, include one or more antibody molecules, one or more aptamer molecules, one or more oligopeptides or small molecules, or various combinations thereof. The cocktail can also include one or more proNGF antagonists, in combination with one or more SorCS2 antagonists.

### Administration

An antagonist or a cocktail of antagonists is administered to a subject as soon as practical after acute myocardial ischemia (AMI) occurs. However, antagonists administered as late as 48 h after the AMI occurs can still be effective. In certain embodiments, an antagonist is administered to the subject within 24 hours, 18 hours, 12 hours, 6 hours or even 2 hours of the AMI. In a specific embodiment, an antagonist is administered initially within 2-6 hours of the AMI. A repeat dose or doses can be administered as appropriate, which can be determined by a skilled physician.

An antagonist can be combined with a pharmaceutically acceptable carrier in any convenient and practical manner, e.g., by admixture, solution, suspension, emulsification, encapsulation, absorption and the like, and can be made in formulations such as tablets, capsules, powder, syrup, suspensions that are suitable for injections, implantations, inhalations, ingestions or the like.

As used herein, a pharmaceutically acceptable carrier includes any and all solvents, dispersion media, isotonic agents and the like. Except insofar as any conventional media, agent, diluent or carrier is detrimental to the recipient or to the effectiveness of the active ingredients contained therein, its use in practicing the methods disclosed herein is appropriate. The carrier can be liquid, semi-solid, e.g. pastes, or solid carriers. Examples of carriers include oils, water, saline solutions, alcohol, sugar, gel, lipids, liposomes, resins, porous matrices, binders, fillers, coatings, preservatives and the like, or combinations thereof.

The concentration of an antagonist in formulations may range from as low as about 0.1% to as much as 15 or 20% by weight and can be selected based on the nature of the particular antagonist used, the mode of administration selected, among other considerations. Thus, a typical formulation for injection could be made up to contain 1 mL sterile buffered water of phosphate buffered saline and 1-1000 mg, possibly 10-100 mg, of an antagonist such as an antibody-based antagonist, for example.

Depending on the nature of the antagonist or the circumstances of AMI, a pharmaceutical formulation containing an antagonist, can be given to the subject by standard routes, including ingestion, or injections via an intravenous, intraperitoneal, subcutaneous, transdermal, intramuscular, intranasal, or sublingual route, or via catheter delivery at the time of percutaneous intervention or during an open heart surgery. Generally speaking, an antibody or aptamer-based antagonist can be delivered intravenously. An RNA-based antagonist can be delivered directly into the heart, e.g., by catheter delivery at the time of percutaneous intervention, or with open heart surgery if bypass grafting is necessary.

The amount of antagonist administered to be effective may depend on the condition of the patient (e.g., age, body weight and health), the time interval since the occurrence of AMI, and the nature of the antagonist. The precise amount of an antagonist to be effective can be determined by a skilled physician.

### EXAMPLES

The present description is further illustrated by the following examples, which should not be construed as limiting in any way. The contents of all cited references (including literature references, issued patents, and published patent applications as cited throughout this application) are hereby expressly incorporated by reference.

### Example-1

This example describes the materials and methods used in the experiments disclosed in Example 2.

### Generation of Inducible Knock-In Mice Misexpressing proNGF

Genomic PAC clone RPCI21-494-C12 was identified from a 129/SvevTACfBR female spleen library purchased from the UK HGMP MRC Geneservice (Cambridge, UK) using standard dot blotting techniques (Osoegawa et al., Genome Research, 10(1):116-128 (2000)). The hemagglutinin (HA) epitope tag was added by site-directed mutagenesis in-frame before the stop codon. The furin recognition site was mutated using site-directed mutagenesis (Stratagene) from KR to AA. A modified neomycin-resistance cassette, consisting of (from 5′ to 3′) frt-loxp-engrailed splice acceptor-pGK-neo^{r}-stop codon-frt-loxp sequences, was inserted 350 bp upstream from the start codon. A diphtheria toxin cassette was used for positive selection. Targeting efficiency was approximately 4%. Three positive independently-derived clones expressing Prongf-HA (called proNGF-HA/+ herein) were expanded and microinjected into C57B1/6 embryos. Three independent lines of proNGF-HA mice were obtained in this manner. Two lines of Ngf-HA knock-in mice (called wtNGF-HA/+) were generated in the same manner, except the furin cleavage site was not mutated in this construct. Southern blotting was performed using standard techniques. PCR was used to identify the knocked-in allele from the wild-type allele by amplifying the HA epitope tag: 5′-TGA AGC CCA CTG GAC TAA ACT T-3′ (SEQ ID NO: 15) and 5′-AAT CTG GAA CAT CGT ATG GG-3′ (SEQ ID NO: 16). The presence of the neomycin-resistance cassette was determined using a second pair of PCR primers: 5′-GAG ATC CAC TAG TTC TAG CCT CGA G-3′ (SEQ ID NO: 17) and 5′-CCC ACA CAC TGA CAC TGT CAC AC-3′ (SEQ ID NO: 18).

### Mouse Breeding and Tissue Processing

All procedures were approved by the Weill Cornell Medical College IACUC. The β-actin-cre delete strain was used to remove the neo-resistance cassette and enable expression of the knocked-in allele. The following primer pairs were used to identify the presence of the cre-recombinase allele: 5′-TTA TAA CAC CCT GTT ACG TAT AGC C-3′ (SEQ ID NO: 19) and 5′-TAT CTC TGA CCA GAG TCA TCC TTA G-3′ (SEQ ID NO: 20). p75NTR^{-/-} mice were purchased from Jackson Laboratories, and backcrossed to >G8 on a C57B1/6 background, prior to using them in proNGF-HA/+ genetic rescue breeding. Other control strains included NGF^{+/-} (Crowley et al., Cell, 76(6):1001-1011 (1994)) and sortilin^{-/-} (Jansen et al., Nat Neuroscience, 10(11):1449-1457 (2007)) mice.

For immunohistochemical analysis, embryonic trunks and adult hearts were snap frozen in OCT:30% sucrose (1:1; Sakura). For protein and RNA analyses, hearts were stored at −80° C. until processing.

FITC-dextran (70 kDa; Sigma) was injected as a bolus through the tail vein of 3 mo mice (Camelleri 1983). After perfusion for 10 minutes, the animals were sacrificed and hearts harvested and processed for immunofluorescence.

### Immunohistochemistry (IHC) and Immunofluorescence (IF)

Frozen cryostat sections were immediately fixed in acetone or in 4% PFA. For IHC, after 15 min in 0.1% H₂O₂/methanol at −20° C., the sections were rinsed in PBS, blocked (5% serum of secondary antibody/0.1% Triton x-100/PBS), and incubated with primary antibody overnight at 4° C. The primary antibodies used were: HA (Sigma, 1:400), p75NTR (ECD: R&D, 1:1000; ICD: Promega, 1:500), sortilin (R&D, 1:400), SorCS2 (ICD: generated by immunizing rabbits against huSorCS2 aa 1138-1159, coupled to KLH, 1:1000; or ECD: R&D, 1:100), activated caspase-3 (Cell Signaling, 1:200), tyrosine hydroxylase (Jackson Immunological, 1:100), phospho-cJun (Cell Signaling; 1:100), CD31 (BD Biosciences, 1:100), CD41 (BD Biosciences, 1:100), fibrin(ogen) (FITC-conjugated, Dako, 1:250), CD68 (TRITC-conjugated, Serotec, 1:100), Iba1 (Wako, 1:400), ICAM (eBioscience, 1:100). Biotinylated secondary antibodies were conjugated to ABC reagent and detected using the VIP kit (Vector Labs). IF was performed essentially as above. Alexa-conjugated secondary antibodies (Invitrogen) were used to detect primary antibodies. Confocal microscopy was performed using either a Zeiss LSM510 or LSM700 laser scanning microscope.

### Immunoprecipitation/Western Blotting

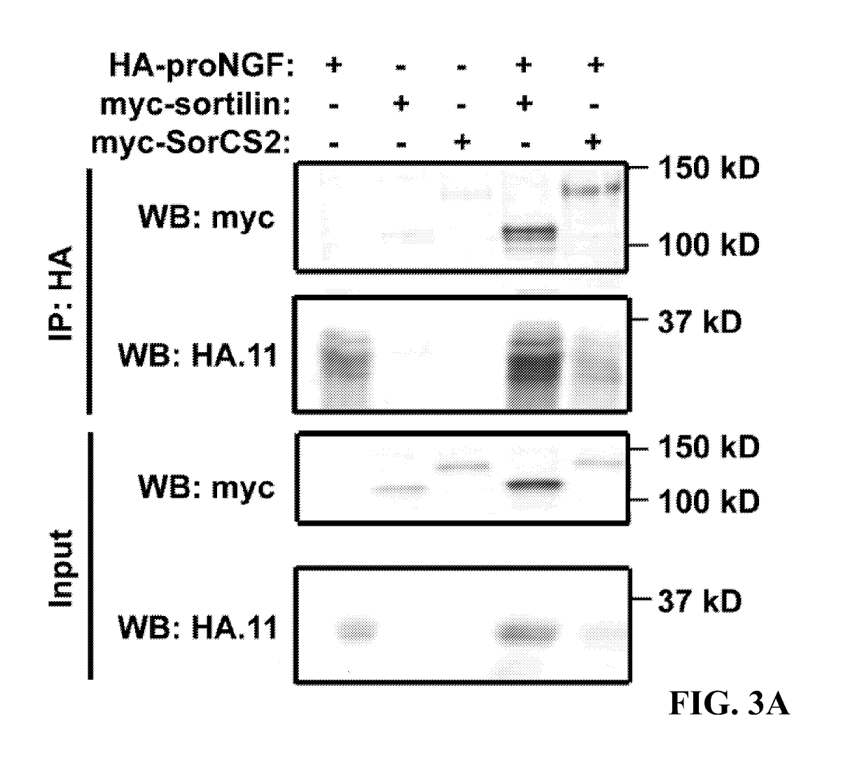

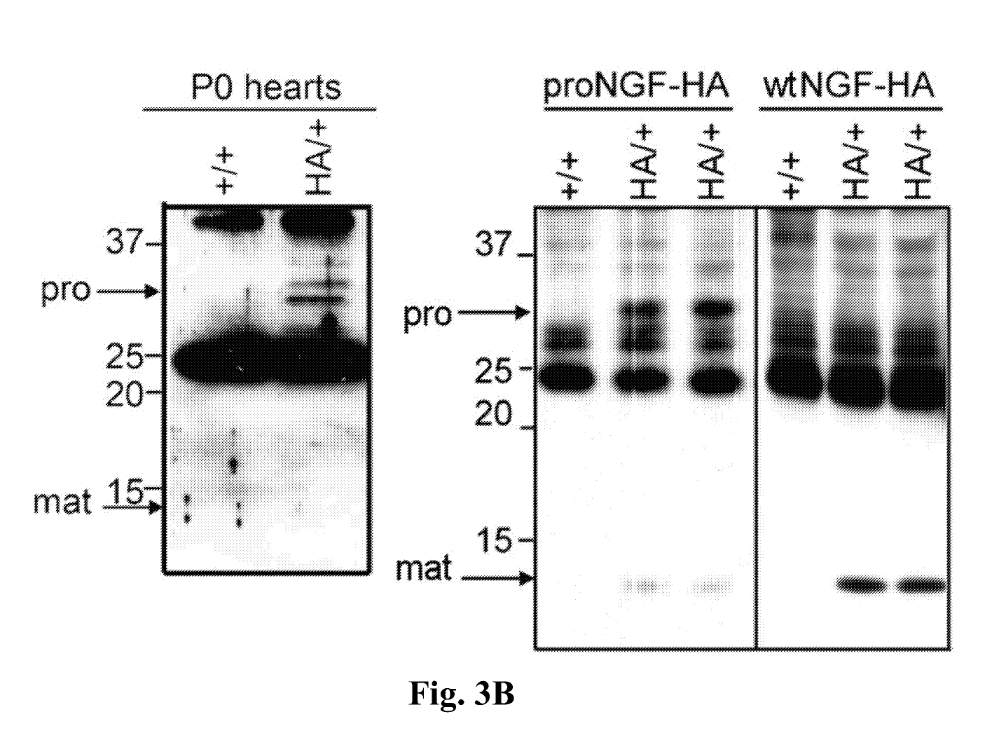

Frozen hearts and brains were homogenized in lysis buffer (0.1M Tris pH 7.4/1% Triton x-100/0.1% NP-40/0.05% SDS/10% glycerol/protease inhibitor cocktail [Sigma]). HA-tagged proteins were pulled down using anti-HA antibody (Sigma) at 4° C. Protein A-sepharose slurry (Sigma-Aldrich) were added to capture the immune-complex, washed extensively, and boiled in SDS-PAGE loading buffer. Blotted proteins were detected using HA.11 monoclonal antibody (Covance) and developed with ECL (Amersham). See FIG. 3b. Transfected 293T cell lysates were immunoprecipitated and Western-blotted using the same method. See FIG. 3a. The myc-tagged sortilin or SorCS2 proteins were detected using a rabbit-anti-myc antibody (Covance).

### RT-PCR

RT-PCR was performed following standard protocols and reagents (Invitrogen). PCR was performed using the HA primers described above, as well as β-actin: 5′-AAA GAG AAG CTG TGC TAT GTT GCT C-3′ (SEQ ID NO: 21) and 5′-GCA TAG AGG TCT TTA CGG ATG TCA A-3′(SEQ ID NO: 22). ELISA.

The NGF Emax kit (Promega) was used for all ELISA measurements, following manufacturer's instructions.

### Transthoracic Echocardiography

An Accuson Sequoia clinical ultrasound equipped with a 14 Hz probe (graciously provided by K. Hajjar, CUWMC) was used for this study. Each mouse was anesthesized using Avertin, allowed to recover baseline heart rate for at least 20 minutes on a warming pad set on low temperature, and depilated. The probe head, whose range was extended using a 1 cm gel offset, was applied to a layer of ultrasound gel on the mouse's chest, and the midline of the heart was detected by the localization of the papillary muscles in the left ventricle. Once the midline was identified, the probe was swept up and down the caudal-rostral axis of the heart to ensure observation at the widest part of the left ventricle, and then the probe was stabilized for 1 min of recording to VHS tape. A printout of the M mode was made, to facilitate calculation of the fractional shortening (% FS) using the following formula:

% FS=[(LVEDD−LVESD)/LVEDD]×100

where LVEDD=left ventricular end-diastolic diameter, and LVESD=left ventricular end-systolic diameter. All statistical analyses were performed using Student's t-test.

### Ischemia-Reperfusion Studies

Anesthesia was induced with 4% inhaled isoflurane and maintained with 2% isoflurane. Mice were then intubated and mechanically ventilated. Core body temperature was monitored with a rectal probe and maintained at 37° C. and ECG was monitored throughout the surgery using a lead II configuration and PowerLab data acquisition system. A left thoractomy was performed in the 4th intercostal space and the pericardium was opened. The left anterior descending coronary artery (LAD) was reversibly ligated with an 8-0 suture for 40 minutes and then reperfused by release of the ligature. Occlusion was confirmed with ST segment elevation on the ECG, regional cyanosis, and wall motion abnormalities. Reperfusion was confirmed by return of color to the myocardium distal to the ligation and disappearance of ST elevation. The suture remained within the wound for identification of the ligature site, and the chest and skin were closed in layers. After surgery, animals were returned to individual cages and given regular food and water for 48 h before euthanasia and tissue harvest. Buprenex (0.1 mg/kg) was administered as needed to ensure that the animals were comfortable following surgery. All surgical procedures were performed under aseptic conditions.

### Electron Microscopy

All reagents were purchased from Electron Microscopy Sciences. Hearts were dissected from mice, rinsed well in cold PBS, and immersed in Karnovsky's fixative (2.5% glutaraldehyde, 4% paraformaldehyde, 0.02% picric acid in 0.1 M PBS) over night. One cm blocks were post-fixed in 1% osmium tetroxide/1.5% potassium-ferricyanide, stained with 1.5% uranyl acetate, and dehydrated through graded ethanol series. After embedding in Spurr's resin, sections were cut at 55-60 nm thickness using a Diatome diamond knife on an RMC MT-7000 ultramicrotome. Sections were contrasted with lead citrate and viewed on a JSM 100 CX-II electron microscope operated at 80 kV. Images were recorded on Kodak 4489 Electron Image film and then digitized at 900 dpi for publication.

### Example-2

This example describes experiments that demonstrate a rapid induction of proNGF and p75NTR following ischemia/reperfusion injury in the murine heart. This example also describes generation of a proNGF knock-in mouse and shows that proNGF acted upon p75NTR-expressing pericytes, leading to endothelial cell activation, enhanced vascular permeability and microvascular compromise, which culminated in a lethal dilated cardiomyopathy in adulthood in the proNGF knock-in mouse. Additionally, this example describes experiments which demonstrate that pericytes ensheathing cardiac capillaries express p75NTR and SorCS2, but not sortilin, during late embryogenesis, establishing that SorCS2, which binds to proNGF, functions as a co-receptor with p75NTR to alter pericyte function.

### Myocardial Ischemia-Reperfusion Induced Expression of proNGF and p75NTR

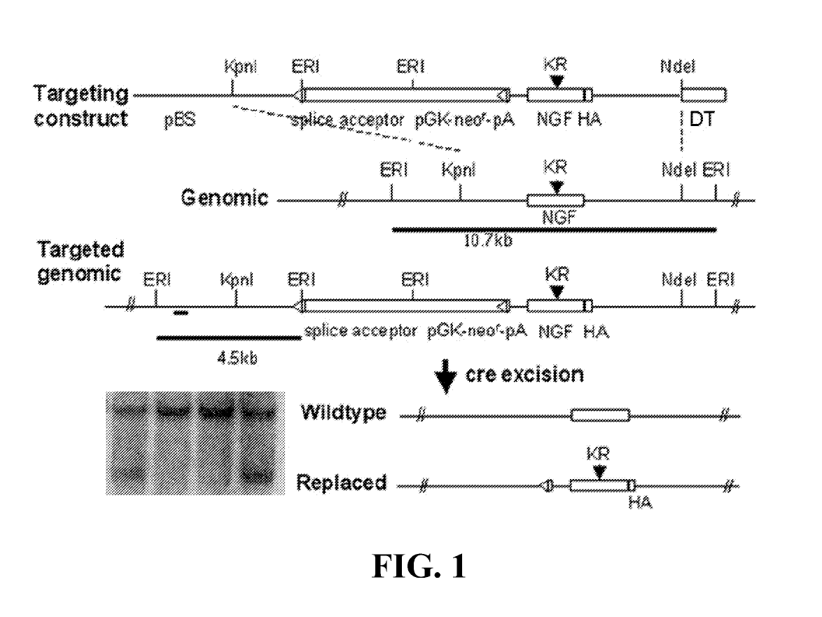

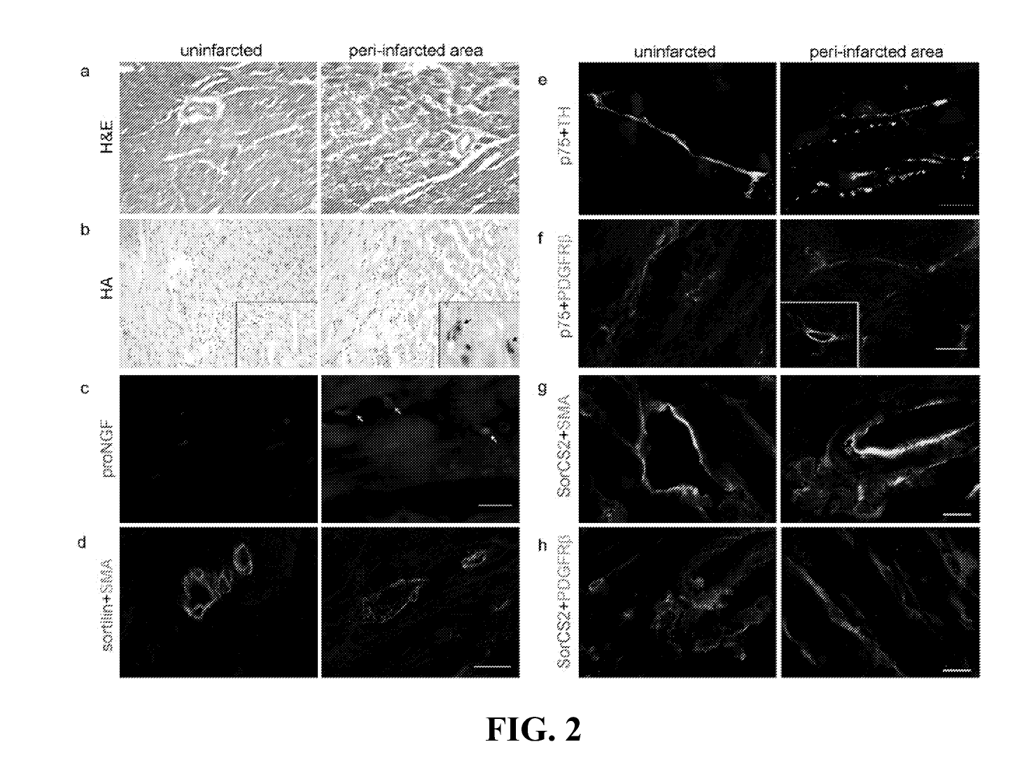

A knock-in mouse was generated in which the Ngf coding exon was replaced with an allele with a C-terminal hemagglutinin (HA) tag to facilitate detection of all forms of NGF (wtNGF-HA/+ mice). This strategy enhanced detection of NGF protein, which is expressed at subnanomolar concentration in the heart. The wtNGF-HA/+ mice were analyzed and were seen to express the knocked-in allele in a manner that was indistinguishable from the endogenous allele (see below and FIG. 1). Adult wtNGF-HA/+ mice were subjected to 40 minutes of occlusion of the left anterior descending coronary artery (FIG. 2a), and injured or sham operated hearts were evaluated for induction of proNGF or mature NGF following 48 hours of reperfusion (I/R injury). In sections of uninjured hearts, low levels of total NGF were detected diffusely in cardiac myocytes, but proNGF immunoreactivity was below the sensitivity of detection (using the HA or a proNGF-specific antibody, respectively; FIGS. 2b and c, left panels). However, I/R injury resulted in an induction of HA immunoreactivity at 48 hours post reperfusion, which was observed in cardiomyocytes and in infiltrating cells in the peri-infarct area (FIGS. 2b and c, right panels). The species of NGF upregulated in this injury paradigm was most likely proNGF, as myocytes in the infarcted region exhibited immunoreactivity using antisera specific for the prodomain of proNGF (Harrington et al., 2004; FIG. 2c). In addition, I/R injury recruited cells into the peri-infarct area that were immunopositive for proNGF (FIGS. 2b and c, right panels, arrows). To evaluate if proNGF receptors were coordinately induced, expression of p75NTR, sortilin, and SorCS2, a sortilin family member that also binds to proNGF (FIG. 3), were evaluated using immunofluorescence in normal and in the I/R injured hearts. Sortilin was expressed by α-smooth muscle actin-positive vascular smooth muscle cells comparably in both uninjured and I/R injured hearts (FIG. 2d). Consistent with prior studies, in the uninjured myocardium p75NTR was expressed exclusively by sympathetic fibers that were immunoreactive for tyrosine hydroxylase (FIG. 2e, left). However, in the peri-infarct regions of I/R injured hearts, p75NTR was expressed on dystrophic axonal processes (FIG. 2e, right) as well as by a subpopulation of infiltrating cells (data not shown). Surprisingly, p75NTR expression was also induced in PDGFRβ-positive pericytes in the peri-infarct region (˜10% of the pericyte population; FIG. 2f, inset), but not by endothelial cells (data not shown). Next expression of sortilin and SorCS2 was evaluated. SorCS2 was also expressed by vascular smooth muscle cells of the larger arterioles in the uninjured myocardium (FIG. 2g) as well as by a subset of hematopoietic cells (data not shown). In the I/R injured hearts, SorCS2 immunoreactivity was maintained in peri-infarct smooth muscle cells (FIG. 2g), and was also detected in resident macrophages and in infiltrating cells (data not shown). Finally, SorCS2 was induced in a subset of PDGFRβ-immunopositive pericytes in the peri-infarct region (˜10-20% of all pericytes; FIG. 2h). Collectively, these results indicate that proNGF and its receptor p75NTR are coordinately induced following ischemia-reperfusion injury.

### Increased Expression of proNGF In Vivo

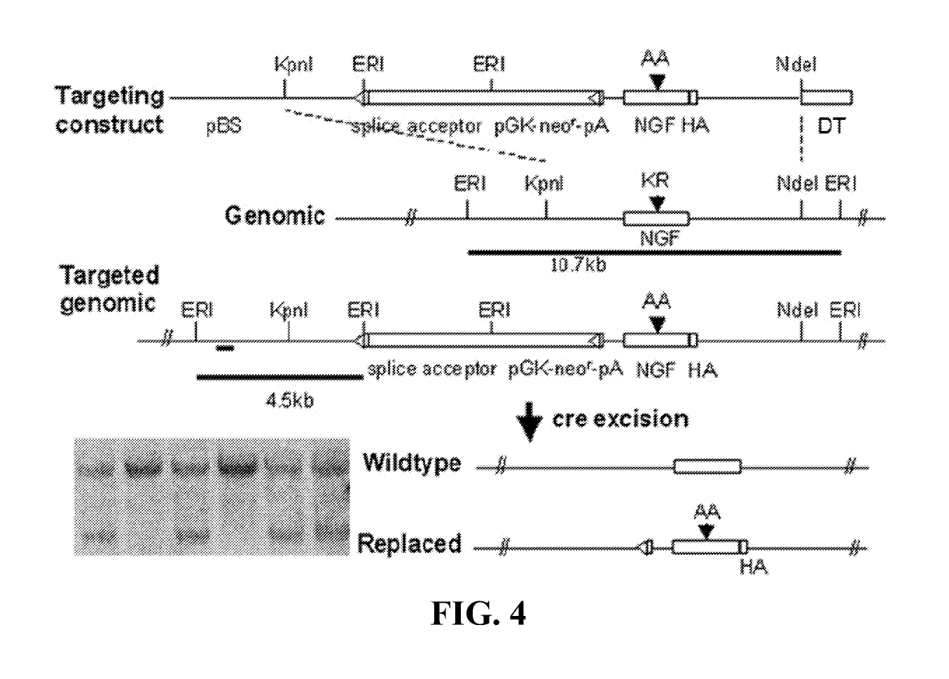

To elucidate the pathophysiological effects of proNGF in the heart, knock-in mice were generated in which the Ngf coding exon was replaced with a mutant allele to impair furin cleavage (aa-K¹²⁰R¹²¹ mutated to AA). In addition, a hemagglutinin (HA) epitope tag was added at the C-terminus to facilitate detection (Prongf-ha) (FIG. 4). Three independently generated lines of mice expressing one allele of Prongf-ha and one endogenous allele (proNGF-HA/+ mice), as well as two independently generated lines expressing one allele of Ngf-ha and one endogenous allele (wtNGF-HA/+ mice), were characterized. wtNGF-HA/+ mice were viable, fertile and indistinguishable from their NGF^{+/+} wild type littermates. Although embryonic lethality of 30-50% was observed in mixed background (129+C57B1/6) proNGF-HA/+ pups, in fully C57B1/6 background mice, surviving pups matured and were fertile in adulthood. As both wtNGF-HA/+ and proNGF-HA/+ mice express one allele of endogenous Ngf, haploinsufficient NGF^{+/-} mice (Crowley 1994) were analyzed in parallel, and serve as a control to evaluate gain-of-function phenotype(s) for proNGF.

Expression of Prongf-ha mRNA and Ngf-ha mRNA was confirmed using RT-PCR of heart tissue from embryonic or neonatal animals. As NGF is expressed at subnanomolar levels in target organs including the heart (Lommatzsch 2005), the inventors were unable to detect HA immunoreactivity (proNGF-HA or wtNGF-HA) in sections of the uninjured neonatal or adult heart (data not shown and FIG. 2). However, HA immunoreactivity was detected in the wtNGF-HA/+ brain, where NGF levels were approximately 2 fold higher than the heart, and NGF was expressed focally in subsets of neurons. ELISA analysis, using antibodies that detect the mature domain and hence both proNGF and mature NGF, indicated that proNGF-HA/+ mice express levels of total NGF protein that were similar to NGF^{+/+} littermates (NGF^{+/+} brain: 141.0+/−8.16 ng/g [average+/−SEM]; proNGF-HA/+ brain: 152.5+/−3.8 ng/g; NGF⁺⁺ heart: 85.2+/−1.8 ng/g; proNGF-HA/+ heart: 84.5+/−5.3 ng/g). To confirm expression of proNGF or mature NGF in the proNGF-HA/+ or wtNGF-HA/+ mice respectively, the HA epitope tag was used to immunoprecipitate all NGF isoforms from heart or brain lysates. Only mature NGF-HA (˜14 kDa=˜13.5 kDa mature NGF+˜0.7 kDa HA) was observed in brain lysates from wtNGF-HA/+ mice. ProNGF-HA (˜35 kDA=˜34 kDa proNGF+˜0.7 kDa HA) was the most highly expressed form observed in proNGF-HA/+ brain lysates, although some cleavage to mature NGF (approximately 10% of total levels) was detectable. In proNGF-HA/+ heart lysates, only proNGF was detectable at P0. See FIG. 3b. These results confirm that mature NGF is the predominant isoform in and secreted by cells in the wtNGF-HA/+ mice, whereas the introduction of a cleavage-resistant proNGF exon significantly abrogates cleavage of proNGF to mature NGF in the proNGF-HA/+ mice.

### ProNGF-HA/+ Mice Exhibited Ventricular Dilation in Adulthood

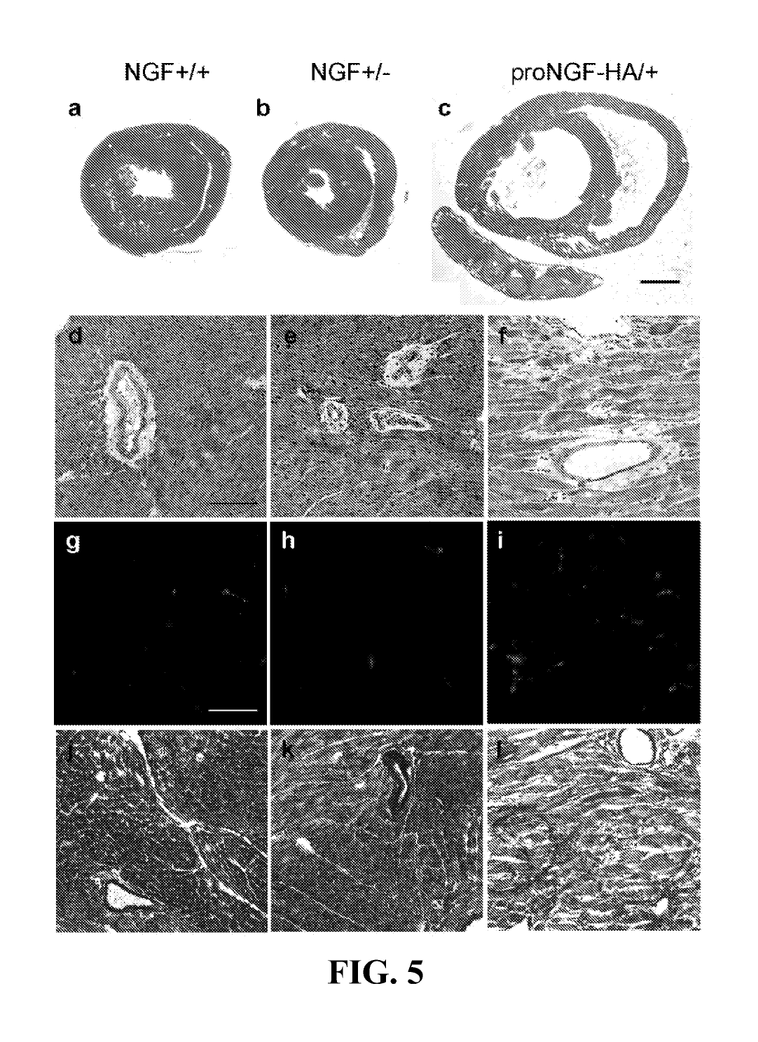

To analyze the effects of proNGF expression in the heart, adult proNGF-HA/+ mice were examined. Marked biventricular enlargement of the hearts was observed in 8 month old (mo) proNGF-HA/+ mice, as compared with NGF^{+/+} hearts in age-matched mice (FIGS. 5a, c). The cardiac chambers of NGF^{+/-} (FIG. 5b) and wtNGF-HA/+ (data not shown) mice were also normal, suggesting that this phenotype reflected expression of proNGF, rather than haplo-insufficiency of mature NGF or an unintentional consequence of targeting this allele. At higher magnification (FIG. 5d-f), an increase of infiltrating cells and focal dropout of myofibrils were observed, predominantly in the subendocardial region of the proNGF-HA/+, but not in NGF^{+/+} or NGF^{+/-}, myocardium. These infiltrating cells expressed the histiocyte marker CD68+ (FIG. 5g-i) or less frequently, were co-labeled with FITC-avidin, a marker for mast cells (data not shown). At 8 mo of age, extensive regions of fibrosis, as evaluated by Masson's trichrome staining, were noted in the subendocardial region of proNGF-HA/+ mice, but not in control animals (FIG. 5j-l). This phenotype is reminiscent of lesions seen in patients with myocarditis and dilated cardiomyopathy (DCM; reviewed in Chihakova 2008).

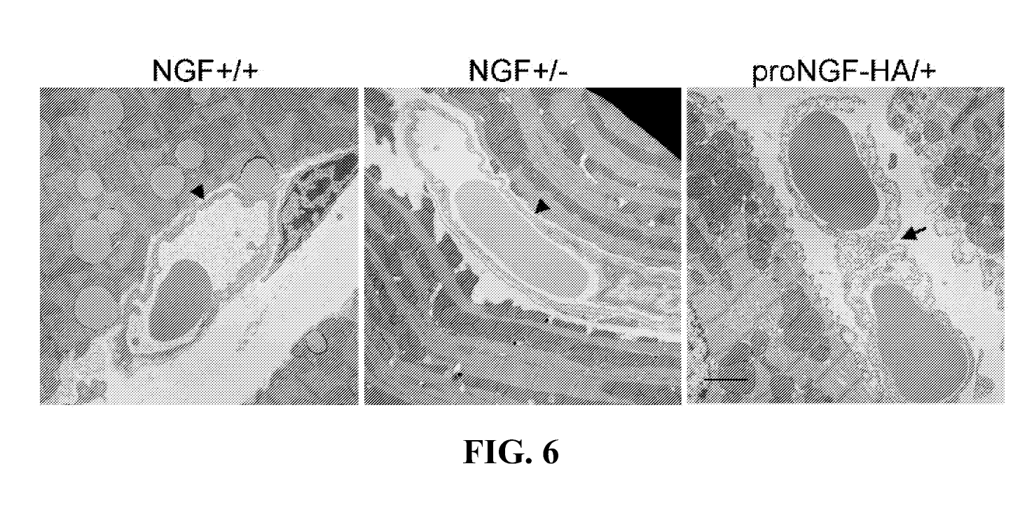

To corroborate the cardiac fibrosis in proNGF-HA/+ mice, cardiac ultrastructure was examined using transmission electron microscopy of mice at 4 months of age (FIG. 6). NGF^{+/+} and NGF^{+/-} hearts appeared healthy, with cardiac myocytes exhibiting intact Z-lines, normal mitochondrial morphology, and lipid droplets. In contrast, proNGF-HA/+ mice displayed focal regions of myofibrillar damage and swollen mitochondria with abnormal cristae, and attenuation of the cytoplasm of endothelial cells with occasional overt rupture and extralumenal erythrocytes. These observations confirmed the light microscopic histological results, and demonstrate a vascular phenotype arising in early adulthood in the proNGF-HA/+ myocardium.

### Genetic Deletion of p75NTR Rescued proNGF-Mediated Cardiomyopathy

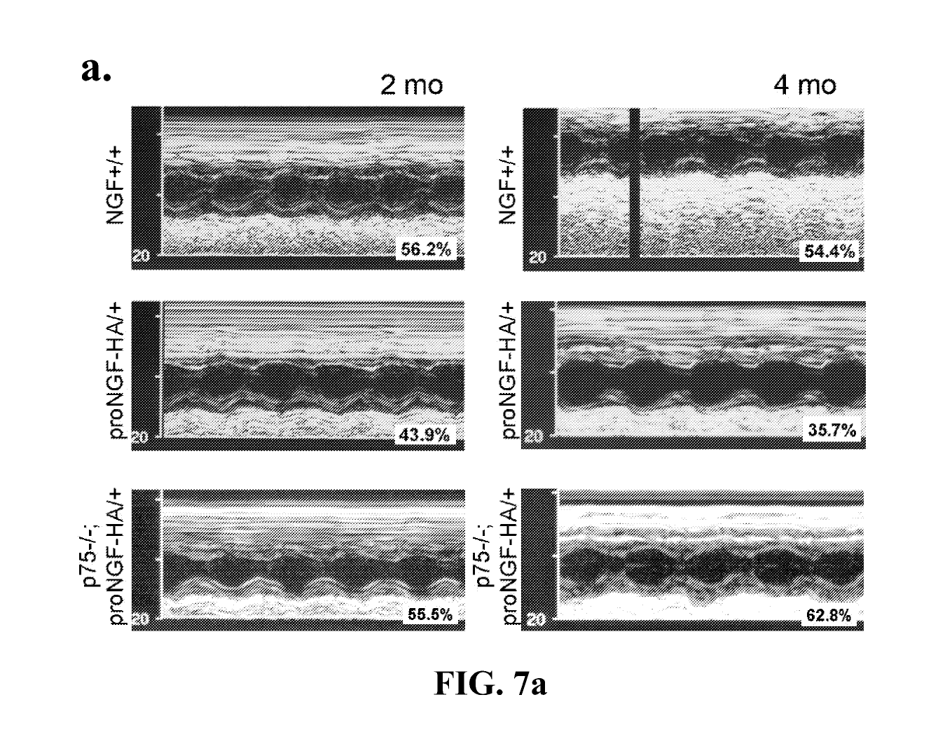

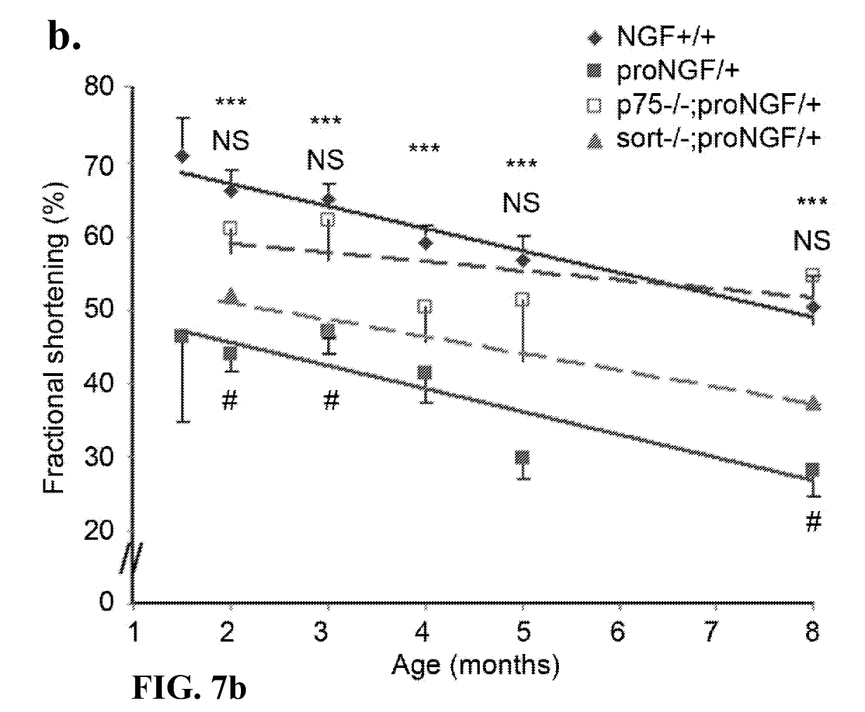

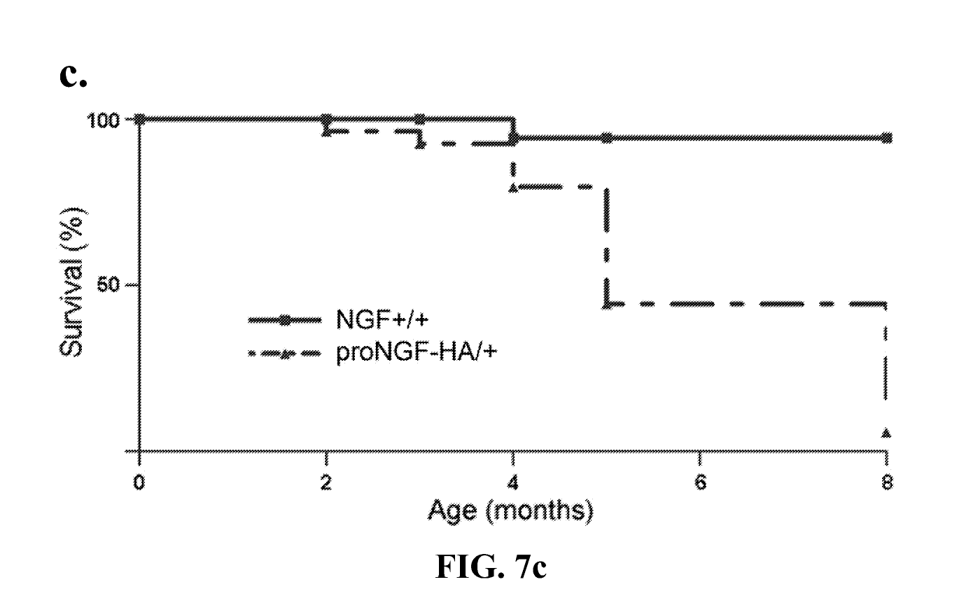

To determine the time course of functional impairment in the proNGF-HA/+mice, transthoracic echocardiography was performed on a cohort of mice beginning at 2 months of age (mo). Functional cardiac compromise was observed in the proNGF-HA/+ mice compared to NGF^{+/+} littermates and to wtNGF-HA/+ knock-in mice, beginning early in life (FIG. 7a are representative traces). Longitudinal analysis demonstrated that significantly impaired contractile function of proNGF-HA/+ mice is apparent by 2 mo and is progressive, as compared to NGF^{+/+} and NGF^{+/-} animals (FIG. 7b, compare NGF^{+/+} [filled blue diamonds] to proNGF-HA [filled red squares]). At all ages analyzed, proNGF-HA/+ mice showed significantly impaired fractional shortening compared with NGF+/+ mice (P<0.005, Student's t-test). For example, at 2 mo of age, the proNGF-HA mice demonstrated fractional shortening (FS) of 44.0% (+/−11.6, average+/−SEM, n=31) compared with NGF+/+ mice at 66.2% (+/−5.3, n=18; P<0.002, Student's t-test). By 5 mo, proNGF-HA/+ mice exhibited 29.9% (+/−2.9, n=15) fractional shortening while NGF^{+/+} mice exhibited 56.9% (+/−3.4, n=14; P<0.002, Student's t-test). It was also observed that proNGF-HA/+ mice were dying at 4-8 months of age. Indeed, Kaplan-Meier analysis showed significant loss of proNGF-HA/+ mice, compared with NGF^{+/+} mice, with 50% of mortality at 4 mo (FIG. 7c). Thus the abnormalities in fractional shortening in the proNGF-HA/+ mice at later times are likely an underestimate of the degree of cardiomyopathy, as most proNGF-HA/+ animals died before 8 months of age.

To determine if the observed cardiomyopathy in proNGF-HA/+ mice was due to activation of established the proNGF receptors, p75NTR and sortilin, the inventors evaluated proNF-HA/+ mice that were deficient for either p75NTR (p75NTR^{-/-}; proNGF-HA/+; n=14) or for sortilin (sort^{-/-}; proNGF-HA/+; n=15). It was hypothesized that, if the observed dilated cardiomyopathy was due to the binding of proNGF to p75NTR and/or to sortilin, a deletion of either receptor for proNGF might prevent this phenotype. Indeed, p75NTR deletion rescued the cardiac hypocontractility as measured by echocardiography (FIG. 7a, representative traces, and b, empty red squares). Even at 8 mo, p75^{-/-}; proNGF-HA/+ fractional wall motion was comparable to NGF^{+/+} hearts (P>0.05 compared with NGF^{+/+} mice, Student's t-test). Concomitant with functional rescue, better survival (identical to NGF^{+/+} mice) and normalized histological appearance of p75NTR^{-/-}; proNGF-HA/+ hearts were observed, as compared with proNGF-HA/+ animals (data not shown).

Surprisingly, sortilin deficiency did not rescue defects in fractional shortening, or histologic evidence of biventricular enlargement and cardiac fibrosis of proNGF-HA/+ mice (FIG. 7b, filled green triangles, and data not shown). Kaplan-Meier survival analysis shows that 50% of sort^{-/-}; proNGF-HA/+ mice died by 4 months of age, and >90% of remaining mice died by 8 mo (data not shown), similar to data obtained with proNGF-HA/+ mice (FIG. 7c). These results indicate either that p75NTR alone was sufficient in mediating proNGF-induced cardiac dysfunction, or that another sortilin family member was the co-receptor in this system. proNGF was observed to bind comparably to sortilin and to SorCS2 as assessed by co-immunoprecipitation of proteins expressed in heterologous cells, indicating that SorCS2 was a relevant co-receptor with p75NTR in the murine heart (FIG. 3). Analysis of wtNGF-HA/+, sort^{-/-}, NGF^{+/-}, and p75NTR^{-/-} mice revealed no significant abnormalities in cardiac wall motion in these mice. Therefore, expression of proNGF, rather than a deficiency of mature NGF, mediated the observed cardiomyopathy.

### Cardiac Dysfunction Began Early in Microvascular Endothelial Cells

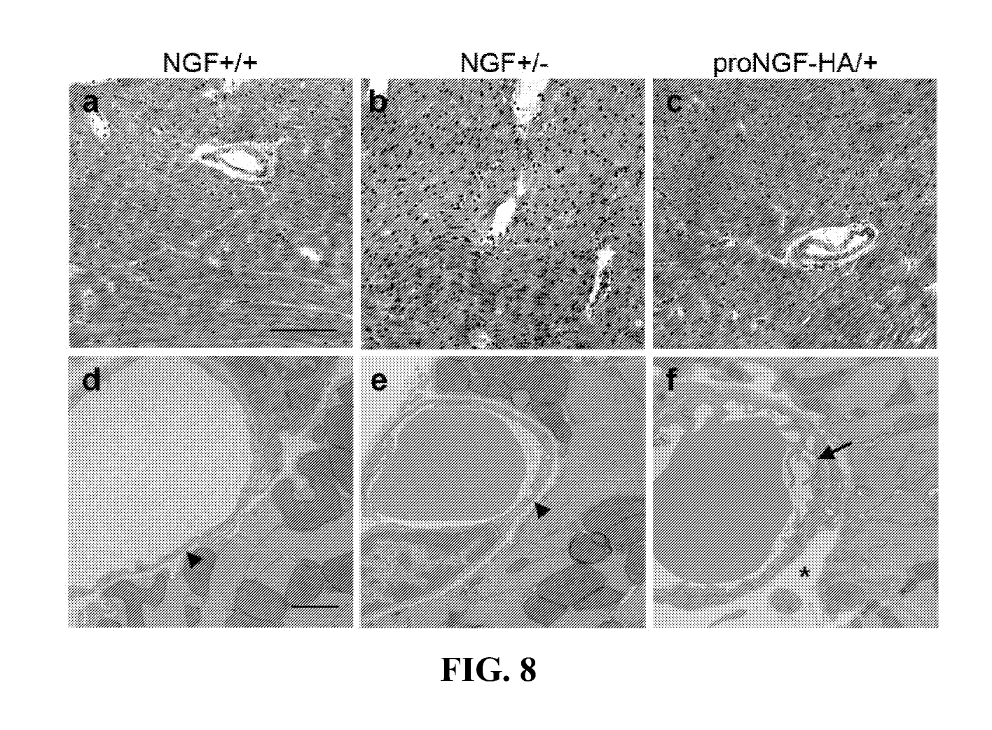

To identify the mechanisms by which proNGF mediates cardiac dysfunction, hearts from young NGF^{+/+} and proNGF-HA/+ mice were analyzed (FIG. 8). At 1 month of age, the NGF^{+/+}, NGF^{+/-}, and proNGF-HA/+ hearts appeared histologically similar to each other (FIG. 8a-c), with intact myofibrils and normal arteries and veins (data not shown). Transmission electron microscopy (TEM) was then performed to analyze cardiac ultrastructure. Hearts from 1 month old NGF^{+/+}, NGF^{+/-}, and proNGF-HA/+ mice exhibited normal cardiac myocyte morphology, with healthy myofibrils and mitochondria. However, in contrast to the hearts in the NGF^{+/+} and NGF^{+/-} mice, proNGF-HA/+ hearts demonstrated microvascular endothelial cells activation, characterized by numerous cytoplasmic vacuoles, and membraneous fronds projecting into the vessel lumen (FIG. 8f, arrow). In evaluation of proNGF-HA/+ mice (N=2), 40/75 microvascular endothelial cells examined in the left ventricle exhibited one or more of these signs of activation. In contrast, these abnormalities were found in less than 5% of microvascular endothelial cells in NGF^{+/+} (N=2) or NGF^{+/-} (N=2) hearts analyzed in parallel. In addition, we observed fibrillar, electron-dense deposits in the lumen of proNGF-HA/+ vessels that morphologically resemble fibrin strands, and we observed increased fibrin(ogen) immunoreactivity in these hearts. Finally, several microvessels in the proNGF-HA/+ heart exhibited perivascular edema with a loss of close apposition of endothelial cells to the extracellular matrix (3/16 vessels examined; for example, see FIG. 8f, asterisk). These results indicate that abnormalities in the cardiac microvascular endothelium of the proNGF-HA/+ mice occurred early in life, and likely resulted in later cardiac fibrosis and dilated cardiomyopathy.

### Microvascular Thrombosis and Loss of Integrity Lead to Dilated Cardiomyopathy in proNGF-HA/+ Mice

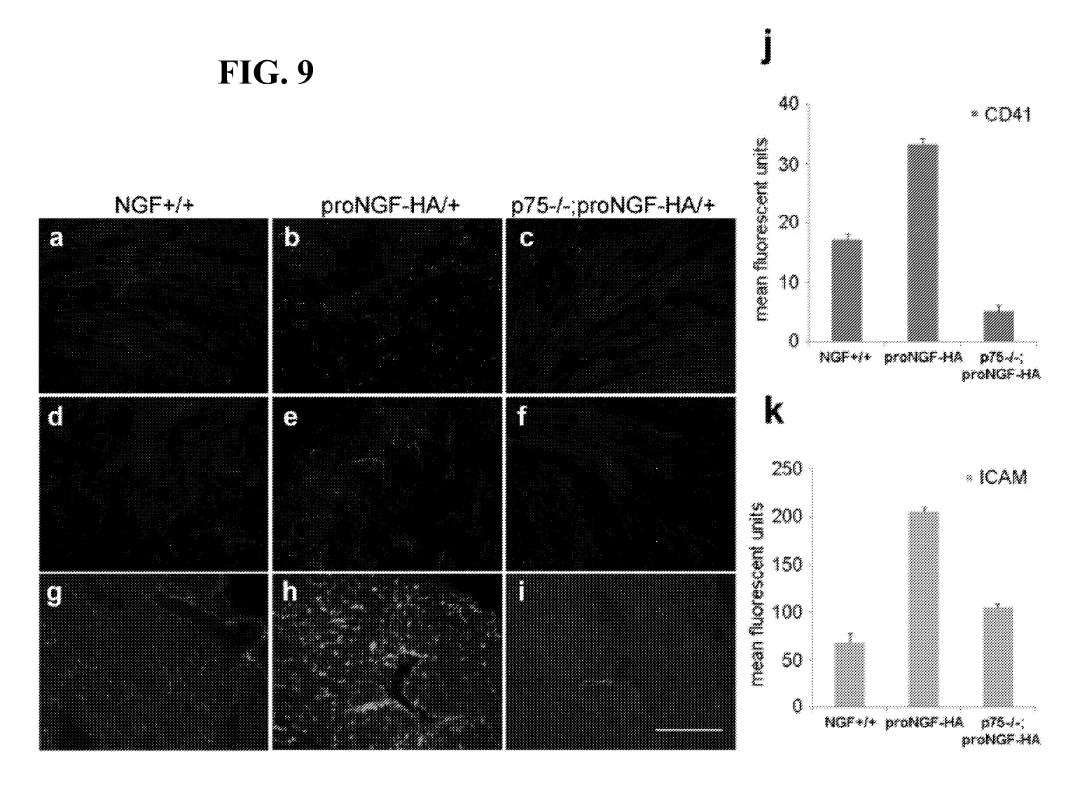

To evaluate potential mechanisms that led to the histological and electron microscopic evidence of microvascular compromise in the young proNGF-HA/+ mice, three parameters were used to assess microvascular integrity. To evaluate whether endothelial cell activation in the proNGF-HA/+ animals led to platelet trapping and fibrin(ogen) deposition, CD41 (to detect platelets) and fibin(ogen) immunoreactivity were assessed. In the proNGF-HA/+ mice, increased CD41 immunoreactivity (FIGS. 9a and b) and increased fibrin deposition were observed in the microvasculature, as compared to wild type littermates. In contrast, p75NTR^{-/-}; proNGF-HA/+ animals showed no increase in platelet (FIG. 9c) or fibrin(ogen) (data not shown) immunoreactivity, indicating that the observed phenotype was dependent upon p75NTR expression. Secondly, ICAM immunoreactivity, an indication of endothelial cell activation, was induced in the proNGF-HA/+ hearts compared with NGF^{+/+} hearts (FIGS. 9d and e), which was again rescued with loss of p75NTR (FIG. 9f), as demonstrated in a semiquantitative analysis (FIGS. 9j and k). Finally, to evaluate whether microvascular permeability was altered in proNGF-HA/+ hearts, perfusion studies using FITC-dextran (70 kD) in 3 mo mice were performed. Extravasation of the FITC-dextran macromolecule was increased in proNGF-HA/+ hearts, in contrast to low levels in the NGF^{+/+} and p75NTR^{-/-}; proNGF-HA/+ hearts (FIG. 9g-i). Taken together, these results indicate that proNGF utilized p75NTR to induce microvascular endothelial cell activation and microthrombi.

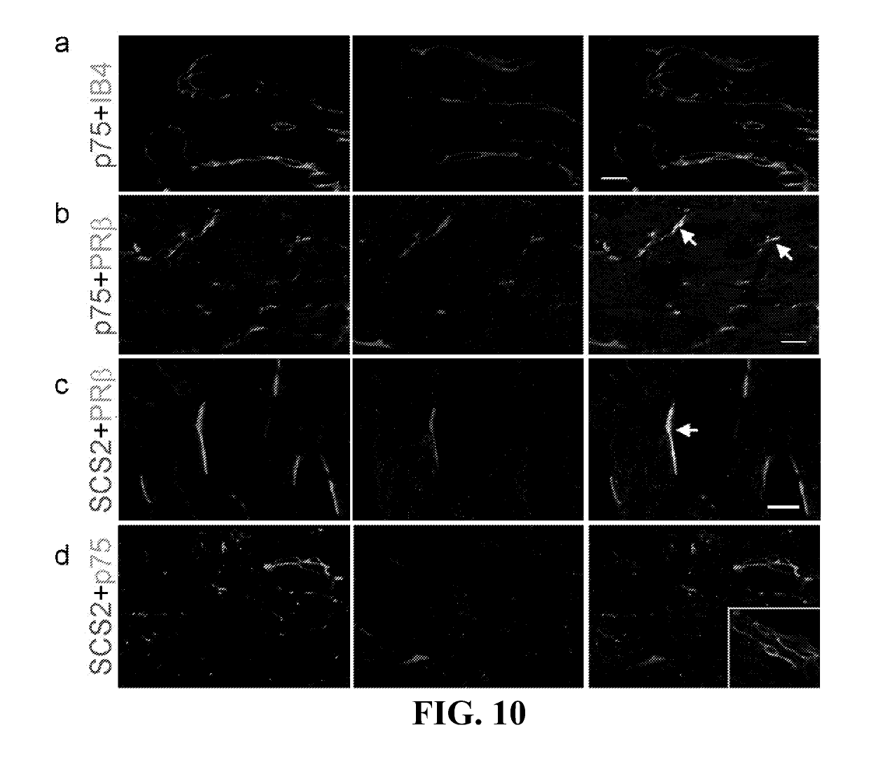

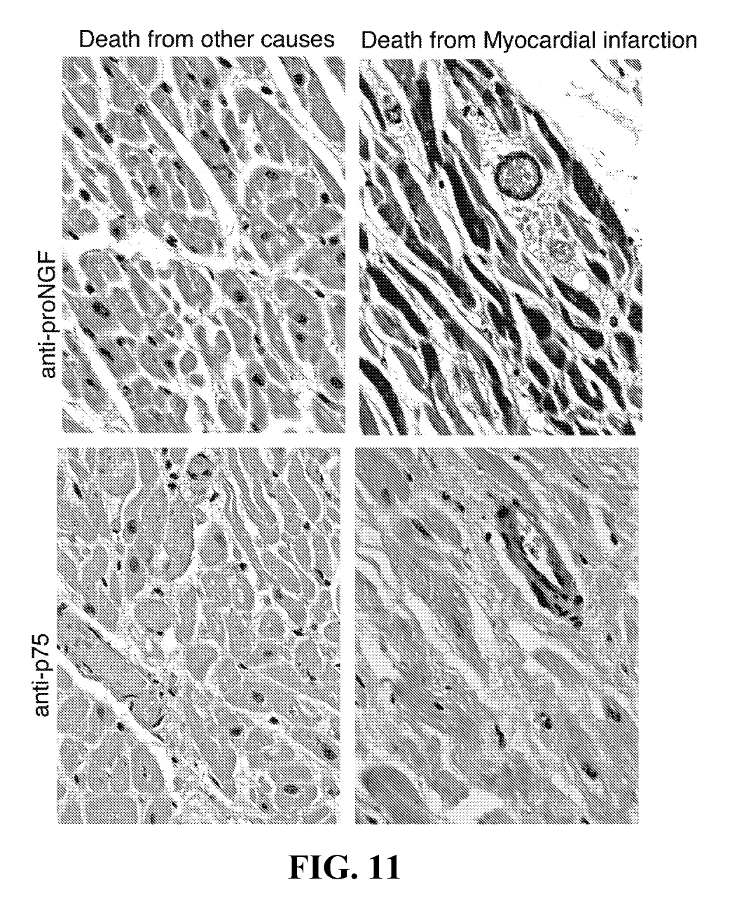

To gain a better understanding of how proNGF:p75NTR signaling led to microvascular damage in proNGF-HA expressing animals, proNGF receptors were localized in the developing and mature heart. The p75NTR receptor was readily detectable on tyrosine hydroxylase (TH)-expressing sympathetic processes extending into the adult NGF^{+/+} and the proNGF-HA/+ heart (FIG. 2d and data not shown). In E17.5 embryos, when sympathetic invasion of the developing heart was minimal, we observed p75NTR expression by the cardiac vasculature. In the wtNGF-HA/+ as well as in the proNGF-HA/+ hearts, p75NTR immunoreactivity co-localized with the pericyte marker, PDGFRβ, in the microvasculature (˜10% of pericyte population; FIG. 10b) but failed to colocalize with isolectin B4-immunopositive endothelium (FIG. 10a and data not shown). In neonatal (data not shown) and adult hearts (FIG. 2f), sortilin was immunolocalized solely to smooth muscle cells of arterioles and arteries, but not to microvascular pericytes. SorCS2, which also binds to proNGF, was detected on a subpopulation of neonatal perivascular cells and colocalized with a subpopulation of PDGFRβ-positive pericytes at P0 (˜10% of pericytes; FIG. 10c), but not with endothelial cells as detected using isolectin B4 (data not shown). Although pericyte populations expressed p75NTR and SorCS2, only rare pericytes co-expressed both receptors at P0 (FIG. 10d, inset). In the adult heart, SorCS2 was localized specifically in the smooth muscle cells of arterioles (FIG. 2g). Collectively, these data show that subpopulations of pericytes express p75NTR or SorCS2 during late embryonic development, and that proNGF expressed by cardiac myocytes induced pericyte dysfunction, leading to microvascular compromise. These results support a model in which impaired pericyte-mediated support of the microvascular endothelium in late gestation and early postnatal life leads to endothelial cell activation, the development of microvascular thrombi and loss of vascular integrity. These effects culminate in microvascular ischemia and cardiac fibrosis, leading to a lethal dilated cardiomyopathy.

### Discussion

To uncover additional roles for proNGF we generated knock-in mice that misexpress the furin-resistant proNGF allele under the endogenous ngf promoter, to elucidate pathophysiological consequences in multiple organs.

A profound progressive deficit has been identified in cardiac contractility in the proNGF-HA/+ mice, which reflect a gain-of-function effect of proNGF, rather than a loss of function effect of reduced mature NGF, as documented by the absence of phenotype NGF haploinsufficient animals. Histological and ultrastructural analyses indicate that the cardiac hypocontractility and fibrosis was initiated by microvascular endothelial activation, leading to increased extracellular matrix deposition and subsequent scarring as well as pro-inflammatory cell infiltration. The pathologic role of proNGF:p75NTR activation in this cascade of tissue damage has been confirmed by genetic rescue of the cardiac phenotype in p75NTR^{-/-} mice. Thus the heart, like the peripheral and central nervous system, utilizes p75NTR as the signaling component of the proNGF receptor complex. Interestingly, sortilin, a co-receptor for p75NTR and proNGF-mediated neuronal effects, did not play a role in this phenotype, since the sort−/−; proNGF-HA/+ genotype provided no rescue to the phenotype. SorCS2, another sortilin family member, has been identified herein as a p75NTR co-receptor in mediating the cardiac phenotype observed herein.

The identification of p75NTR+ pericytes, rather than endothelial cells, during embryonic cardiac development provides a surprising mechanism for proNGF-mediated cardiomyopathy in the young adult mouse. It is postulated herein that proNGF, secreted by cardiac myocytes, acts locally on p75NTR+ pericytes, to induce pericyte dysfunction, leading to a lack of trophic support of the microvascular endothelium.

### Example-3

This example illustrates how to develop and characterize a human antibody to the prodomain of proNGF, and demonstrate that the antibody is specific for proNGF but not related neurotrophins. This example also illustrates preclinical models to show that an anti-proNGF antibody blocks proNGF-induced cell death, and promotes cardioprotection in animal models of myocardial ischemia. This example further describes design of a small clinical study of patients with an acute myocardial infarction for establishing that delivery of anti-proNGF limits infarct size and promotes cardiac recovery.

### Pre-Clinical Studies

ProNGF and the prodomain thereof can be produced recombinantly. Recombinant prodomain can be used as an immunogen to generate antibodies specific for the prodomain and hence specific for the proNGF. The antibodies can be analyzed for their efficacy of neutralization of activities of the recombinant proNGF.

High avidity monoclonal antibodies specific to different regions of the prodomain can be generated and identified. Both the pro-domain and mature domain of NGF are highly conserved across species. As shown in FIG. 12, significant regions of identity are present within the pro-domain of NGF from human to mouse, enabling the generation of a spectrum of antibodies to the prodomain directed to different regions, motifs or tertiary structures of the prodomain.

Antibodies that are specific for the prodomain of proNGF, without recognizing the prodomains of other human proneurotrophins, or the mature domains, are documented. The prodomain of proNGF is clearly distinct from those of other proneurotrophins (FIG. 13), making it unlikely that antibodies raised against the prodomain of proNGF will cross-react with other family members, or with mature NGF. Nevertheless, the specificity of anti-proNGF antibodies can be verified by (i) modified two-sites ELISA (ii) co-immunoprecipitation and (iii) uptake assays.

ELISA—To identify proNGF antibodies that interact only with proNGF but not other proneurotrophins or other growth factors that are known to play roles in cardiovascular cells, commercially available ELISA kits for NGF, BDNF, NT-3, NT-4, FGF, and VEGF will be modified by substituting the coating antibody with anti-proNGF in these sandwich ELISA assays. This approach is scalable and adaptable to positively identify a range of candidate proNGF antibodies and to rule out cross-reactivity with other growth factors.

Co-immunoprecipitation—The ability of the anti-proNGF monoclonal antibodies to recognize human, murine, potentially porcine or primate proNGF can be assessed to confirm utility in preclinical models. This can be performed using the media of cells expressing proNGF from other species, and co-immunoprecipitation analysis with the candidate monocloncal antibodies.

Uptake Assay—After identifying a panel of anti-proNGF antibodies, the antibodies are assessed in their abilities in blocking proNGF binding and internalization using HT1080 cells that co-express p75^{NTR} and sortilin or SorCS2. Recombinant proNGF can be labeled with Alexa-594 and uptake assay can be carried out as recently described (Feng et al., J Mol Biol. 396:967, 2010). Anti-proNGF antibodies can be added to the culture medium 30 min before the proNGF addition. The amount of internalized Alexa-conjugated proNGF can be quantified 24 hrs later by semi-automated fluorescent microscopy as documented (Feng et al., J Mol Biol. 396:967, 2010). This assay has been developed for high through-put screening. As a control, comparable studies can be performed with Alexa-conjugated mature NGF. The anti-prodomain antibody should immunodeplete proNGF, but not mature NGF.

Cell Death Assay—Once anti-proNGF antibodies have been identified that block proNGF uptake, their utility in blocking proNGF-induced cell death can be verified using HT-1080 cells that co-express p75 with sortilin or SorCS2, and with primary superior cervical ganglion (SCG) neurons. This latter model has been extensively utilized to document the pro-apoptotic properties of the proneurotrophins (see Lee, Science, 294: 1945-48 (2001); Teng et al., J Neurosci, 25:5455 (2005); Yano et al., J Neurosci., 29: 14790 (2009), for example).

Antibody optimization would be carried out including affinity maturation and half-life extension.

Preclinical analysis of anti-proNGF efficacy. Studies of the biotherapeutic potential of anti-proNGF are performed in cardiac ischemia/reperfusion models in rodents, and subsequently extended to porcine/dog or primate models. Current polyclonal anti-proNGF antibodies could serve as a control.

In brief, animals are intubated, and subjected to 40 minutes of coronary ischemia by occlusion of the left anterior descending artery, followed by reperfusion. Anti-proNGF antibodies are delivered beginning at 2 or 6 hours following re-perfusion (to coincide with delivery times achievable in humans). Animals are dosed daily (or as optimal based on pharmacological studies). Ongoing studies that quantitate proNGF levels at 1, 3, 7, 10 days post ischemia/reperfusion injury in rodents can guide the duration of treatment. Three methodologies are used to determine efficacy. First, a cohort of animals is sacrificed at 3 or 10 days, and blood and cardiac tissue are analyzed for levels of proNGF (by ELISA and Western blot), and the shed ectodomains of p75 and sortilin/SorCS2 (a marker of ligand binding and receptor activation), as well as microvascular markers (by Western blot). Second, a cohort of rodents is sacrificed at 3 or 10 days, and hearts are examined histologically. This enables an examination of infarct size, and protection of the microvasculature (morphometric evaluation). Lastly, a cohort of rodents is imaged using Doppler-flow echocardiography at 3 and 10 days post ischemia reperfusion to assess the functional effects of anti-proNGF delivery on regional wall motion abnormalities (as compared to equivalent concentrations of isotype matched non-immune IgG). Comparable studies are undertaken on a larger mammal (pig) which is typically used for cardioprotection studies.

As an additional analysis, studies are performed on the proNGF “knock-in” mouse model. Immunoneutralization of proNGF should prevent the progressive cardiomyopathy observed in this animal. For these studies, a cohort of proNGF expressing mice is treated with anti-proNGF antibodies for two months (age 2-4 months) and Doppler imaging is performed to document improved cardiac function, and histological analysis is undertaken to document microvessel preservation and reduced expression of proNGF.

### Translational Studies

Assessment of plasma levels of proNGF in humans post myocardial ischemia. Total NGF levels in human plasma have been detected, consistent with prior reports. A quantitative ELISA can be developed using anti-prodomain antibodies to determine the magnitude of proNGF induction in human post-MI, using banked specimens or in a prospective study conducted at Weill Medical Center. Blood can be collected at presentation to the emergency room, immediately post-stenting/dilation, and then at 24 hour intervals for 4 days, and then at follow-up at 1 and 2 weeks. This analysis can be extended to a “biomarker panel” assessing the levels of shed ectodomain of p75, as an immediate endpoint for the clinical trials, as well as conventional assessment of cardiac injury (troponin). It should be noted that p75 ectodomain are present at low levels in human plasma. In preclinical models, the shed ectodomains increase following ligand binding.

### Clinical Approach

Patient selection. Only patients with no prior history of myocardial infarction are considered (no prior medical history, or prior normal stress test or cardiac imaging), and no other significant co-morbidities, and have onset/duration of positive symptoms of less than 6 hours (window of 2-6 hours permitted). Patients must be evaluable by cardiac catheterization, and amenable to stent placement or dilation (no contraindications).

Patient evaluation and drug delivery. Utilizing cardiac catheterization, patients must demonstrate a reduction in ejection fraction of at least 15%, with an identifiable coronary lesion, regional wall motion abnormalities, no sustained atrial or ventricular tachyarrythmias, and restoration of flow with stent placement/dilation. Ideally, drug (anti-proNGF) or control can be delivered within 4-8 hours following reperfusion.

PK/PD studies. Biomarkers are evaluated every 6 hours for 24 hours, then daily for 4 days, then at 1 and 2 weeks following delivery. This includes plasma for proNGF (ELISA), shed ectodomain of p75.

Analysis of efficacy. Patients are evaluated by cardiac MRI, echocardiography to assess functional wall motion abnormalities, and recovery of function at 2 and 4 weeks post-delivery.

What is claimed is:

1. A method of limiting microvascular damage following acute myocardial ischemia in a subject, comprising administering to the subject a proNGF antagonist.

2. The method of claim 1, wherein said proNGF antagonist is an antibody specific for proNGF which inhibits the binding of proNGF to p75^{NTR} and/or SorCS2.

3. The method of claim 2, wherein said antibody is an antibody directed to the pro-domain of proNGF.

4. The method of claim 1, wherein said proNGF antagonist is a nucleic acid or peptide aptamer which specifically binds to proNGF and inhibits the binding of proNGF to p75^{NTR} and/or SorCS2.

5. The method of claim 1, wherein said proNGF antagonist is an oligopeptide or small molecule which inhibits the binding of proNGF to p75^{NTR} and/or SorCS2.

6. The method of claim 6, wherein said nucleic acid molecule is an anti-sense molecule or siRNA which reduces the level or activity of proNGF mRNA.

7. The method of claim 1, wherein said proNGF antagonist is administered to the subject within 48 hours of acute myocardial ischemia.

8. The method of claim 1, wherein the proNGF antagonist is administered to the subject via ingestion, injection, catheter delivery during percutaneous intervention, or directly to the heart during open heart surgery.

9. A method of limiting microvascular damage following acute myocardial ischemia in a subject, comprising administering to the subject a SorCS2 antagonist.

10. The method of claim 9, wherein said SorCS2 antagonist is an antibody directed to SorCS2 which blocks the binding of proNGF to SorCS2.

11. The method of claim 10, wherein said antibody is an antibody directed to the ectodomain domain of SorCS2.

12. The method of claim 9, wherein said SorCS2 antagonist is a nucleic acid or peptide aptamer which binds to SorCS2 and blocks the binding of proNGF to SorCS2.

13. The method of claim 9, wherein said SorCS2 antagonist is an oligopeptide or small molecule compound which inhibits the interaction of SorCS2 with proNGF and/or p75NTR.

14. The method of claim 9, wherein said SorCS2 antagonist is an anti-sense molecule or siRNA which reduces the level or activity of SorCS2 mRNA

15. The method of claim 9, wherein said SorCS2 antagonist is administered to the subject within 48 hours of acute myocardial ischemia.

16. The method of claim 9, wherein SorCS2 antagonist is administered to the subject administered to the subject via ingestion, injection, catheter delivery during percutaneous intervention, or directly to the heart during open heart surgery.
